SEQUENCE LISTING (54 sequences)

SEQ ID NO: 1 - DNA, 20 bp, Homo sapiens
ctccacccaagggaacttgt

SEQ ID NO: 2 - DNA, 22 bp, Homo sapiens
taggaccaagaagacctgcatc

SEQ ID NO: 3 - DNA, 20 bp, Homo sapiens
cccaaagagcgatgtggact

SEQ ID NO: 4 - DNA, 20 bp, Homo sapiens
cttctagcctgctggtgtcc

SEQ ID NO: 5 - DNA, 20 bp, Homo sapiens
cgactgttgcctctcgtaca

SEQ ID NO: 6 - DNA, 20 bp, Homo sapiens
gaggaggttcacagcccttt

SEQ ID NO: 7 - DNA, 21 bp, Homo sapiens
tgtgtgtggcttttcaaacct

SEQ ID NO: 8 - DNA, 20 bp, Homo sapiens
gtacgattgtgcgggctgat

SEQ ID NO: 9 - DNA, 20 bp, Homo sapiens
agttcgaggaaccctagtga

SEQ ID NO: 10 - DNA, 20 bp, Homo sapiens
ttgtagtggatcgtgcctcg

SEQ ID NO: 11 - DNA, 20 bp, Homo sapiens
aaagtacccacactgcgagg

SEQ ID NO: 12 - DNA, 20 bp, Homo sapiens
cttcgcgtcccagatgcttc

SEQ ID NO: 13 - DNA, 18 bp, Homo sapiens
atcttcgctgtcgtcctt

SEQ ID NO: 14 - DNA, 20 bp, Homo sapiens
gaagccaagaatctccgtag

SEQ ID NO: 15 - DNA, 20 bp, Homo sapiens
ctggggttcttcctgccatt

SEQ ID NO: 16 - DNA, 21 bp, Homo sapiens
atggcaaggattgaagggtgt

SEQ ID NO: 17 - DNA, 21 bp, Homo sapiens
cagtcactgagagcatcctca

SEQ ID NO: 18 - DNA, 21 bp, Homo sapiens
cctgatggagcactggtttgc

SEQ ID NO: 19 - DNA, 22 bp, Homo sapiens
acaacccaggcaagttccacca

SEQ ID NO: 20 - DNA, 18 bp, Homo sapiens
gccagccagaccacagcg

SEQ ID NO: 21 - DNA, 20 bp, Homo sapiens
ccagagcctgaagaagccaa

SEQ ID NO: 22 - DNA, 20 bp, Homo sapiens
gagccttccacgtttagagc

SEQ ID NO: 23 - DNA, 20 bp, Homo sapiens
agggtgacagcattctctgc

SEQ ID NO: 24 - DNA, 20 bp, Homo sapiens
ccggtggcttgtttctctta

SEQ ID NO: 25 - DNA, 22 bp, Homo sapiens
ggcaaaaggatggtgacatgaa

SEQ ID NO: 26 - DNA, 20 bp, Homo sapiens
ttttcgccttgctgttgctg

SEQ ID NO: 27 - DNA, 21 bp, Homo sapiens
gacatttcacacacgcagtcg

SEQ ID NO: 28 - DNA, 20 bp, Homo sapiens
acattttgggaaggcaggct

SEQ ID NO: 29 - DNA, 20 bp, Homo sapiens
atgcagtggggtcaaatcgt

SEQ ID NO: 30 - DNA, 20 bp, Homo sapiens
tgttcgtcacactgggcttt

SEQ ID NO: 31 - DNA, 20 bp, Homo sapiens
ggcttcgcaaggagagatca

SEQ ID NO: 32 - DNA, 20 bp, Homo sapiens
gaatgcctgcaatgtcgtcc

SEQ ID NO: 33 - DNA, 18 bp, Homo sapiens
acacgacatcttccagta

SEQ ID NO: 34 - DNA, 18 bp, Homo sapiens
caccttgttcacctcatt

SEQ ID NO: 35 - DNA, 20 bp, Homo sapiens
ctggcgctatgatgaccaca

SEQ ID NO: 36 - DNA, 23 bp, Homo sapiens
gaaataggatgcaccatcagacc

SEQ ID NO: 37 - DNA, 21 bp, Homo sapiens
ccccacattcctgaggatgag

SEQ ID NO: 38 - DNA, 19 bp, Homo sapiens
gggctgggaaatggtcctg

SEQ ID NO: 39 - DNA, 20 bp, Homo sapiens
tcgtctctgcgaggtcaaag

SEQ ID NO: 40 - DNA, 20 bp, Homo sapiens
tgctgttgtccgctcctaag

SEQ ID NO: 41 - DNA, 20 bp, Homo sapiens
acgcatggcttaggacgctc

SEQ ID NO: 42 - DNA, 20 bp, Homo sapiens
gtcacccacgagttcccgag

SEQ ID NO: 43 - DNA, 22 bp, Homo sapiens
ctgtctagtggttcttgttggt

SEQ ID NO: 44 - DNA, 22 bp, Homo sapiens
gggtgttggtggtgtagatatt

SEQ ID NO: 45 - DNA, 19 bp, Homo sapiens
gctcgtgggacattcgtga

SEQ ID NO: 46 - DNA, 22 bp, Homo sapiens
accacatccagcctgttttgtt

SEQ ID NO: 47 - DNA, 19 bp, Homo sapiens
caccagagtggccctcgaa

SEQ ID NO: 48 - DNA, 22 bp, Homo sapiens
atcttccacttcacctgccgtc

SEQ ID NO: 49 - DNA, 22 bp, Homo sapiens
gaacaatgtcttaccaccagca

SEQ ID NO: 50 - DNA, 21 bp, Homo sapiens
agtcctgatgactgggaaacc

SEQ ID NO: 51 - DNA, 23 bp, Homo sapiens
acctcttcacacagtctaactcg

SEQ ID NO: 52 - DNA, 23 bp, Homo sapiens
tcagttcaccttcattcacctca

SEQ ID NO: 53 - DNA, 20 bp, Homo sapiens
ggctcccagacagcgggcaa

SEQ ID NO: 54 - DNA, 18 bp, Homo sapiens
cggaactgaaactgacac

(19) United States
(12) Patent Application Publication
(10) Pub. No.: US 2020/0315996 A1
(43) Pub. Date: Oct. 8, 2020

(54) Treatment of Endometriosis and Niclosamide Derivatives

(71) Applicant: Board of Trustees, Southern Illinois University, Carbondale, IL (US)

(72) Inventors: Kanako Hayashi, Carterville, IL (US); James MACLEAN, II, Carterville, IL (US); Mandy KING, Murphysboro, IL (US)

(73) Assignee: Board of Trustees, Southern Illinois University, Carbondale, IL (US)

(21) Appl. No.: 16/303,758

(22) PCT Filed: May 26, 2017
(86) PCT No.: PCT/US17/34730

§ 371 Date: Nov. 21, 2018

Related U.S. Application Data
(60) Provisional application No. 62/344,960, filed on Jun. 2, 2016.

(51) Int. Cl. A61K 31/167; A61K 9/00; A61P 15/00

(52) U.S. Cl. CPC A61K 31/167; A61P 15/00; A61K 9/0053

(57) ABSTRACT

Niclosamide is an effective therapeutic medicament for the treatment of endometriosis. Niclosamide treatment can reduce the size of endometriotic lesions and reduce cell proliferation in the lesions. Niclosamide treatment can result in the inhibition of proteins and genes associated with inflammatory signaling, including those associated with NFκB and STAT3 activation, but without altering the expression of steroid hormone receptors. Niclosamide can be effective in reducing the growth of endometriotic lesions in female mice without impairing their ability to become pregnant and without negative effect on gestation and offspring. The invention herein is useful in treating symptoms associated with endometriosis, such as reducing the growth of lesions of the endometrium, without disrupting normal reproductive function in females. The invention also relates to derivatives of niclosamide having improved solubility and enhanced efficacy.

### CROSS-REFERENCE TO RELATED APPLICATIONS

This application claims benefit to provisional U.S. Patent Application No. 62/344,960, filed Jun. 2, 2016, entitled “Niclosamide for the Treatment of Endometriosis,” which is herein incorporated by reference in its entirety.

### FIELD

The present invention relates to methods of treating endometriosis and to alleviating symptoms associated with the disease, particularly the reduction of endometrial lesions and endometrial pain, by the application of niclosamide to target inflammatory mechanisms without disrupting female reproductive function. The disclosure also provides compounds that are derivatives of niclosamide that can be useful in such methods.

### BACKGROUND

Endometriosis affects 6-10% of women of reproductive age [Giudice, 2010]. Although endometriosis is a benign disorder, approximately 50% of affected women experience severe chronic pelvic pain and infertility [Eskenazi, 1997; Meuleman et al., 2009]. Long-term treatment of patients with chronic pelvic pain associated with endometriosis involves repeated courses of therapy: surgical, medical or both [Kennedy, 2005].

Endometriosis is gynecological disease characterized by the presence of endometrial or endometrium-like tissue, comprising both glandular epithelium and stroma, outside the uterine cavity (e.g. endometriotic lesions) [Giudice, 2004; Bulun, 2009]. It can be a benign gynecological disorder, which, in a sub-population of female patients, can develop into an aggressive disease. Endometriosis is associated with various distressing symptoms including dysmenorrhoea, dyspareunia, pelvic pain and reduced fertility.

Current strategies for the treatment of endometriosis only temporarily relieve the symptoms of the disease (i.e., pain and reduction or elimination of endometrial tissue outside the uterine cavity of the subject). Laparoscopic surgery provides temporary pain relief, but the recurrence rate is conservatively estimated to be 50% after five years [DeCherney et al., 1992; Evers et al., 1991; Giudice et al. 2004].

The most widely used medical drugs are oral contraceptives, gonadotropin-releasing hormone receptor (GnRH) agonists and progestins, which suppress ovarian function, and reduce pelvic disease and associated pain [Giudice, 2010; Agarwal et al., 2002]. However, these hormonal treatments are often of limited efficacy, elicit side-effects, temporarily inhibit fertility, and ultimately result in high recurrence rates of symptoms.

GnRH agonist therapy carries a significant risk of bone loss due to the induced hypoestrogenic state [Al Kadri, 2009], and has a 50% or higher rate of recurrence of symptoms within 2 years [Practice Committee of American Society for Reproductive Medicine, 2008; Waller, 1993]. Progesterone resistance commonly arises as a major complication to progestin therapy, leading to escalation of estrogen function [Kim, 2013a]. Although hysterectomy with oophorectomy can be the best treatment, it elicits irreversible fertility loss. Therefore, there is a need to identify therapeutic targets and efficient drugs that are improvements over current treatment options for the treatment of endometriosis.

Although nonsteroidal anti-inflammatory drugs, such as ibuprofen, have also been used for the treatment of endometriosis, these drugs primarily relieve dysmenorrhea, without affecting the progression of the disease. Moreover, such relief tends to be incomplete and short-lived.

Endometriosis is defined as the presence of endometrium-like tissue, consisting of proliferating endometrial glands and stroma, outside the uterine cavity, primarily on the pelvic peritoneum and ovaries [Giudice, 2004; Bulun, 2009]. Major molecular distinctions in endometriotic lesions are overproduction of estrogen, prostaglandins and cytokines [Bulun, 2009; Burney, 2012].

Estrogen enhances the survival and persistence of endometriotic lesions, whereas prostaglandins and cytokines mediate pain, inflammation and infertility [Giudice, 2004; Bulun, 2009; Burney, 2012].

Remarkably, increased macrophage, prostaglandin, cytokine and chemokine contents have been found in the peritoneal fluid from endometriosis patients [Burney, 2012; Berkkanoglu, 2003; Rana, 1996]. This milieu of cytokines and growth factors creates a microenvironment that encourages endometrial cell attachment, invasion and vasculogenesis [Burney, 2012; Berkkanoglu, 2003; Rana, 1996].

Chemokines play a major role in the recruitment of macrophages to the site of endometrial tissue engraftment in the peritoneal cavity, a critical step for endometriotic growth and progression [Capobianco, 2013]. Thus, inflammatory environment further enhances inflammation, and consequently promotes endometriotic cell survival and growth [Gonzalez-Ramos, 2012a].

One of the key features in endometriosis is the overproduction of estrogen, which can subsequently accelerate the growth of endometriotic lesions [Bulun, 2009; Burney, 2012]. While systemic estrogen can be a player for endometriosis, local estrogen production by aromatase and development of an inflammatory environment in the presence of prostaglandins and cytokines are hallmarks of the progression of endometriosis [Giudice, 2004; Bulun, 2009; Burney, 2012; Attar, 2009].

Cytokines and growth factors that have been implicated in proinflammatory environment in endometriosis are up-regulated by NFκB signaling [Gonzalez-Ramos, 2012a; Gonzalez-Ramos, 2012b]. Aberrant STAT3 activation enhances the etiology of endometriosis [Kim 2005; Okamoto, 2015], and its activation is synergistically increased when endometrial stromal cells are cocultured with macrophages while the inflammatory environment is developing [Itoh, 2013]. Similarly, NFκB activation is also increased via modulation of cytokines and growth factors [Gonzalez-Ramos, 2012a; Gonzalez-Ramos, 2012b]. Niclosamide suppresses abnormal cellular processes by targeting NFκB and STAT3 signaling in cancer cells [Jin, 2010; Ketola, 2012; Khanim, 2011; Kim, 2013b; Li, 2013a; Li, 2013b].

It has been reported that abundant LEP (leptin) levels are observed in serum and peritoneal fluid from endometriosis patients, and endometriotic tissues [Lima-Couy, 2004; Matarese, 2000; Vigano, 2002; Wu, 2002; Wu, 2010]. LEP promotes proliferation, migration and invasion in endometriotic cells through JAK2/STAT3 signaling [Ahn, 2015; Oh, 2013]. Ablation of LEP signaling disrupts endometriotic growth and progression in mouse model [Styer, 2008]. MUC13 is also known to promote NFκB activity, and further enhances epithelial cell inflammation [Sheng, 2013].

Overproduction of prostaglandins (PG) via PTGS2 is also a feature of endometriosis [Burney, 2012]. High levels of local estrogen and PGE2 are maintained in endometriotic lesions by positive feedback mechanisms: PGE2 activates steroidogenic proteins and SYP19A1 (aromatase) leading to local estrogen biosynthesis [Attar, 2009], and estrogen induces PTGS2, which in turn stimulates PGE2 production [Bulun, 2009].

Despite the large number of women who suffer from severe chronic pain and infertility related to endometriosis, current treatments do little more than temporarily relieve the symptoms of the disease but abolish fertility.

Niclosamide, or 5-chloro-N-(2-chloro-4-nitrophenyl)-2-hydroxybenzamide, is an efficacious, minimally toxic and FDA-approved anti-helminth drug that has been used in patients for decades [Al-Hadiya, 2005; Andrews, 1982]. The anti-parasitic activity of niclosamide was originally reported to be mediated by inhibition of mitochondrial oxidative phosphorylation and anaerobic ATP production [Weinbach, 1969].

Recently, several groups have reported that niclosamide disrupts multiple signaling pathways including NFκB, STAT3 and WNT signaling in cancer models [Jin, 2010; Ketola, 2012; Khanim, 2011; Kim, 2013b; King, 2015; Li, 2013a; Li, 2013b; Osada, 2011; Sack, 2011; Wieland, 2013]. Importantly, niclosamide shows extremely low toxicity and side-effects, as treatment of normal cells and animals with near pharmacological doses elicits no toxic effects [ORGANIZATION WH, 2002]. Thus, niclosamide could be an inhibitor of not only cancer but also endometriosis progression and growth by targeting these signaling mechanisms.

Niclosamide has been orally administered for the treatment of intestinal helminthic infections. One of the features of niclosamide is low toxicity as shown when it was evaluated by World Health Organization (WHO) and Food and Agriculture Organization of the United Nations (FAO) in 1988, and published in “Data Sheet on Pesticides, No. 63, Niclosamide” (WHO/VBC/DS/88.63) [ORGANIZATION WH, 2002]. The toxicity kinetics of niclosamide administered orally in rats for 4 weeks elicits no adverse effects up to 2000 mg/kg daily. Acute toxicity in mice is reported as LD50=>1500 mg/kg b.w. (body weight). Similarly, niclosamide treatment in dogs is safe at doses up to 4500-6000 mg/day for 4 weeks. No signs of intoxication have been observed in humans treated at 1000 mg/day. However, it has been reported that nausea and abdominal pain occurs in only 10% of human patients following an oral dosage of 2000 mg/day.

Previous analyses of niclosamide toxicity have ignored the potential impact on fertility.

Niclosamide's limitations as a drug include poor water solubility, minimal oral bioavailability (10%), and low plasma half-life (7 h) in rats [Chang, 2006]. Although there has been increased interest in niclosamide's action against key pathological pathways as an anti-cancer drug; low solubility, low bioavailability and poor pharmacokinetic profile result in variation of its anti-cancer efficacy when it is used in clinical trials.

The inventions described herein provide methods for using niclosamide that are improvements over current treatment options for the treatment of endometriosis. The invention also relates to derivatives of niclosamide for inhibiting inflammatory pathways without disrupting reproductive functions in endometriosis, cancer, and other diseases. [Prather 2016]

### SUMMARY

Despite the large number of women who suffer from severe chronic pain and infertility related to endometriosis, current treatments temporarily relieve the symptoms of the disease but abolish fertility. In the present disclosure, we report that the FDA-approved small molecule, niclosamide, can inhibit the growth and progression of endometriotic lesions using an established mouse model [Han, 2012; Hirata, 2005; Kulak, 2011; Pelch, 2012].

The present study showed that niclosamide did not affect steroid hormone receptors in the endometriotic lesions. In the present study, we also report that niclosamide is effective at suppressing the activation of NFκB and STAT3 signaling in the endometriotic lesions. Niclosamide treatment did not disturb ovarian function in the recipient mice, suggesting that niclosamide does not inhibit local estrogen function in the endometriotic lesions or systemic estrogen production and function in the ovary.

Our transcriptional profiling also indicated that many of the genes regulated by niclosamide were linked to inflammatory responses. Lep, a proinflammatory cytokine, and Muc13, a transmembrane mucin glycoprotein, were significantly downregulated in the endometriotic lesions by niclosamide treatment. On the other hand, niclosamide did not inhibit PTGS2 (COX2) expression in the endometriotic lesions. The inhibitory mechanisms of niclosamide did not target estrogen and prostaglandin production and function.

While niclosamide might not directly inhibit hormone action, niclosamide can effectively disrupt the inflammatory environment. In addition, niclosamide can alter the behavior of endometriotic cells through modulation of the NFκB and STAT3 signaling pathways including expression of their associated downstream target genes.

Previous analyses of niclosamide toxicity have ignored its potential impact on fertility. In our studies, the mice receiving niclosamide treatment exhibited normal estrous cyclicity and successfully conceived while undergoing the treatment. Furthermore, treatment of niclosamide did not induce preterm birth, impact fetal development, and normal postnatal growth curves were observed. These results suggest that niclosamide does not cause toxic effects, and effective doses do not disrupt reproductive functions. Nevertheless, niclosamide maintained the reduction of size of endometriotic lesions. Thus, drug re-purposing of niclosamide could provide a rapidly-distributed, potential therapy without major side effects for the treatment of endometriosis patients.

The present disclosure shows that niclosamide did not affect steroid hormone receptors in the endometriotic lesions. Niclosamide did not disturb ovarian function in the recipient mice, suggesting that niclosamide does not inhibit local estrogen function in the endometriotic lesions or systemic estrogen production and function in the ovary.

Current treatments for endometriosis temporarily inhibit fertility. However, niclosamide treatment did not disturb normal reproductive function in mice. The present invention provides that niclosamide can be a potential therapeutic drug for the treatment of endometriosis by targeting inflammatory mechanisms while preserving normal fertility.

The present invention also provides derivatives of niclosamide that can be useful in identifying targets of niclosamide and as improved therapeutics for endometriosis and for other disorders for which niclosamide is currently used as a therapeutic.

### BRIEF DESCRIPTION OF THE SEQUENCE LISTING

The invention can be more fully understood from the following detailed description and the accompanying Sequence Listing, which form a part of this application.

The sequence descriptions summarize the Sequence Listing attached hereto. The Sequence Listing contains standard symbols and format used for nucleotide sequence data comply with the rules set forth in 37 C.F.R. § 1.822.

### BRIEF DESCRIPTION OF THE DRAWINGS

The foregoing and other aspects, features, and advantages of the present disclosure will be better understood from the following detailed description taken in conjunction with the accompanying figures, all of which are given by way of illustration only, and are not limitative of the present description in which:

FIGS. 1A and 1B show the experimental design of studies performed to assess the effect of oral niclosamide on mice with implanted endometriotic lesions.

FIG. 1A is a schematic showing the design of the experimental design of the study (Study 1) to assess the effect of niclosamide on endometriotic lesions implanted in mice. Endometriotic lesions were implanted in female mice. After three days, the mice were treated daily with niclosamide at 0, 100, or 200 mg/kg of body weight. After 24 days of niclosamide treatment, the endometriotic lesions were harvested and assessed.

FIG. 1B is a schematic showing the design of the experimental design of the study (Study 2) to assess the effect of niclosamide on endometriotic lesions implanted in the mice, the fertility of the mice, and the offspring of the mice. The mice received endometriotic lesion implants or were subjected to sham surgery. After three days, the mice were treated daily with niclosamide at 0, 100, or 200 mg/kg of body weight. After 4 days of niclosamide treatment, the mice were bred. Niclosamide treatment was continued until the pups were born; then, the niclosamide treatment was discontinued. Twenty-one days after the birth of the pups, the endometriotic lesions were harvested and assessed. E: embryonic day; PND21: postnatal day 21.

FIGS. 2A and 2B show assessments of endometriotic lesions.

FIG. 2A shows microscopic images of endometriotic lesions examined after 3 weeks of treatment at doses of 0, 100 or 200 mg/kg b.w./day of niclosamide. Upper panels show sutured endometriotic lesions in situ, which have adhered to the peritoneal walls of implanted mice, and vascularization (arrows) in the recipient mice. Middle panels show GFP positive lesions were observed under fluorescent light. Bottom panels show the endometriotic lesions isolated from the recipient mice.

FIG. 2B shows histological sections of endometriotic lesions. The tissues were stained with hematoxylin and eosin. Upper panels show endometriotic lesions attached to peritoneal wall tissues. Middle and bottom panels show intact epithelial and stromal cells of the endometriotic lesions were observed in control and niclosamide treated mice. (LE: indicates luminal epithelium; S: indicates stroma).

FIGS. 3A-3B show the effect of niclosamide on the growth of endometriotic lesions. Uterine tissue was harvested from donor mice and implanted into the peritoneal walls of recipient mice. After three weeks of daily treatment with niclosamide at 0, 100, or 200 mg/kg of body weight, the endometriotic lesions was harvested from the recipient mice, and the size of the endometriotic lesions was evaluated.

FIG. 3A is a plot showing the mean weight (g) of lesions obtained from the recipient mice.

FIG. 3B is a plot showing increases in the growth of implanted lesions obtained from the recipient mice, expressed as fold change in size.

FIG. 4 shows images displaying the effect of niclosamide on proliferation, apoptosis, angiogenesis and steroid hormone receptors in endometriotic lesions in mice treated with niclosamide at 0, 100, or 200 mg/kg of body weight. Immunohistochemical analysis was performed on endometriotic lesions were screened to show the expression of: MKI67 (Ki67), a marker of cellular proliferation; cellular apoptosis as shown by TUNEL analysis; endothelial cell marker PECAM1 (CD31); estrogen receptor ESR1; and progesterone receptor PGR.

FIG. 5 shows the effect of niclosamide on the expression of proteins involved in inflammatory signaling in endometriotic lesions. Immunohistochemical analysis of endometriotic lesions shows the expression of p-CHUK (IKK); p-STAT; NOS2 (iNOS); and PTGS2 (COX2).

FIGS. 6A-6C show graphs quantifying the relative expression levels of mRNA for genes related to inflammation. After receiving endometriotic lesions, implanted mice were treated with niclosamide daily at 0 or 200 mg/kg of body weight for 3 weeks. The endometriotic lesions were harvested and the expression of mRNAs for many genes associated with inflammation were assessed by RNA sequencing. Of the 951 genes whose relative mRNA expression was originally analyzed, qPCR analysis confirmed the differential expression of a variety of genes.

FIGS. 7A-7G show the effect of niclosamide on fertility. Following transimplantation or sham surgeries, and daily treatment with niclosamide at 0 or 200 mg/kg body weight, the mice were mated with male mice. Niclosamide treatments continued until pups were born. Different parameters of pregnancy and procreation were assessed.

FIGS. 7A-7E shows graphs quantifying: the average amount of time that passed (days) before vaginal plugs formed in mated mice; the length of time the mice gestated (days), the average amount of time between mating and subsequent birth of young; the average number of pups born to the litters of the mated mice; the average weight (g) of pups at birth; and the average weight (g) of pups at three weeks after their birth, on post-natal day 21 (PND21), respectively.

FIG. 7F shows images of the morphology and volume of endometrial lesions in the post-gestational mice, determined on post-natal day 21. The mice received endometriotic implants or underwent sham surgery, and then received niclosamide at 0 or 200 mg/kg of body weight as treatment for 21 days.

FIG. 7G is a graph showing increases in the growth of implanted endometriotic lesions harvested from recipient mice on post-natal day 21, expressed as fold changes in size.

FIG. 8 shows the protocol for the chemical synthesis of derivatives of niclosamide conjugated to biotin.

FIGS. 9A-9G shows derivatives of niclosamide designed for improved water solubility.

FIG. 9A shows niclosamide further modified with a replacement moiety R, which shows the site of modification for proposed niclosamide derivatives.

FIG. 9B shows a replacement moiety for R that was obtained by acidification of the O-ethylamino group of Intermediate 3 of FIG. 8 with HCl to provide a protonated salt form that displays significantly improved water solubility (˜2 mM), approximately 100 times higher than niclosamide (<20 μM (ORGANIZATION WH, 2002)).

FIGS. 9C-9G shows replacement moieties for R for synthesizing specific derivatives of niclosamide.

### DETAILED DESCRIPTION

The following detailed description is provided to aid those skilled in the art. Even so, the following detailed description should not be construed to unduly limit, as modifications and variations in the embodiments herein discussed can be made by those of ordinary skill in the art without departing from the spirit or scope of the present specification.

### I. Abbreviations and Short Forms

The following abbreviations and short forms are used in this specification.

“b.w.” means body weight

“CHUK” means conserved helix-loop-helix ubiquitous kinase

“COX2” means cyclooxygenase-2

“DNA” means deoxyribonucleic acid

“ESR1” means estrogen receptor 1

“IKK” means IκB kinase

“iNOS” means inducible nitric oxide synthase

“GFP” means green fluorescent protein

“GnRH” means gonadotropin-releasing hormone receptor

“MKI67” means Marker of Proliferation Ki-67

“NFκB” means nuclear factor kappa-light-chain-enhancer of activated B cells

“PCR” means polymerase chain reaction

“PECAM” means platelet endothelial cell adhesion molecule

“PGR” means progesterone receptor

“PTGS” means prostaglandin-endoperoxide synthase

“RNA” means ribonucleic acid

“SEM” means standard error of the mean

“STAT” means signal transducer and activator of transcription

### II. Definitions

Unless defined otherwise, all technical and scientific terms used herein have the same meaning as commonly understood by those of ordinary skill in the art to which the disclosure pertains. Units, prefixes, and symbols can be denoted by their accepted SI form. Provision, or lack of the provision, of a definition for a particular term or phrase is not meant to signify any particular importance, or lack thereof. Rather, and unless otherwise noted, terms used and the manufacture or laboratory procedures described herein are well known and commonly employed in the art. Conventional methods are used for these procedures, such as those provided in the art and various general references. The following definitions are provided to aid the reader in understanding the various aspects of the present disclosure.

In the context of the present invention and the associated claims, the following terms have the following meanings:

The articles “a” and “an” are used herein to refer to one or to more than one (i.e., to at least one) of the grammatical object of the article. By way of example, “an element” means one element or more than one element.

As used herein, the term “about” is a flexible term with a meaning similar to “approximately” or “nearly.” The term “about” indicates that exactitude is not claimed, but rather a contemplated variation. Thus, as used herein, the term “about” means within 1 or 2 standard deviations from the specifically recited value, or a range of up to 20%, up to 15%, up to 10%, up to 5%, or up to 4%, 3%, 2% or 1% compared to the specifically recited value.

The term “antibody” refers to a polypeptide comprising a framework region from an immunoglobulin gene or fragments thereof that specifically binds and recognizes an antigen. Antibodies can be polyclonal or monoclonal or derived from serum.

The term “control” means the level of a molecule, such as a protein or nucleic acid, normally found in nature under a certain condition. In certain conditions, a control level of a molecule can be measured in an animal, tissue, cell, or specimen that has not been subjected, either directly or indirectly, to a treatment. A control level is also referred to as a wildtype or basal level. These terms are understood by those of ordinary skill in the art.

The term “diestrus stage” refers to a state or interval of sexual inactivity or quiescence.

The term “expression” or “expressing” refers to the production of a functional product, such as an RNA transcript or an endogenous DNA sequence. The term can also refer to a protein or polypeptide.

The term “gene” refers to a nucleic acid molecule that encodes a particular protein, or in certain cases, a functional or structural RNA molecule.

The term “kit” as used herein, is used in reference to a combination of therapeutics and other materials. It is not intended that the term “kit” be limited to a particular combination of therapeutics and/or materials.

The term “nucleic acid” means a polynucleotide (or oligonucleotide) and includes single- or double-stranded polymers of deoxyribonucleotide or ribonucleotide bases. Nucleic acids can also include fragments and modified nucleotides.

The term “protein” and “polypeptide” are used synonymously to mean any peptide-linked chain or polymer of amino acids, regardless of length or post-translational modification, e.g., glycosylation or phosphorylation.

The term “subject” refers to an individual that is the subject of treatment, observation, or experiment. In some embodiments, it encompasses mammals with endometriosis or endometriotic lesions. “Subject” includes, but is not limited to, rodents, non-human primates, and humans. It is preferred that a “subject” be free of tape worms or other helminth infections.

The term “therapeutically effective amount or dose” means an amount of active compound or pharmaceutical agent that elicits the biological or medicinal response in a tissue, system, animal or human that is being sought by a researcher, veterinarian, medical doctor or other clinician, which includes alleviation or palliation of the symptoms of the disease being treated.

The term “treating” means reversing, alleviating, inhibiting the progress of, or preventing endometriosis, or one or more symptoms associated with the disorder.

“qPCR” refers to quantitative polymerase chain reaction, a laboratory technique for measuring a targeted DNA molecule

“TUNEL assay” refers to a method for detecting DNA fragmentation that results from apoptosis

Although compositions, antibodies, kits, and methods similar or equivalent to those described herein can be used in the practice or testing of the present invention, suitable compositions, kits, and methods are described below. All publications, patent applications, and patents mentioned herein are incorporated by reference in their entirety. In the case of conflict, the present specification, including definitions, will control. The particular embodiments discussed below are illustrative only and not intended to be limiting.

### II. Overview of Several Embodiments

In one embodiment, the invention relates to a method for treating endometriosis comprising administering to a subject in need thereof a therapeutically effective amount of niclosamide.

In some embodiments, the method is administered to a subject that is a mammal.

In some embodiments, the method is administered to a subject that is pregnant or capable of becoming pregnant. In some embodiments, the subject is an adolescent or adult human subject.

In some embodiments, the method is associated with reduction of endometrial lesions in the subject. In other embodiments, the method is associated with reduction of endometrial pain in the subject. In preferred embodiments, the method of treatment of endometriosis is associated with reducing one or more symptoms associated with endometriosis.

In some embodiments, the niclosamide is administered orally. In further embodiments, the method comprises orally administering the niclosamide in the form of a capsule, a sachet, a tablet, a syrup, an elixir, or a lozenge.

In some embodiments, the niclosamide is administered in a daily dosage of about 100-200 mg/kg of body weight. In particular embodiments, the niclosamide is administered in a daily dosage of about 200 mg/kg of body weight.

In some embodiments, the niclosamide is administered to the subject for at least three weeks.

In another embodiment, the invention relates to a method for the treatment of endometriosis which comprises administering niclosamide to a subject in an amount sufficient to reduce or eliminate endometrial tissue outside the uterine cavity of the subject.

In some embodiments, the treatment results in significant reduction in the expression of MKI67, but does not result in significant reduction of the expression of estrogen receptor or progesterone receptors, in endometrial lesions. In other embodiments, the treatment results in significant reduction in the expression of p-CHUK, p-STAT3, and NOS2, but does not result in significant reduction of the expression of COX2, in endometrial lesions. In other embodiments, the treatment inhibits the expression of p-CHUK, p-STAT3, and/or NFκB or STAT3 in endometrial lesions.

Another embodiment of the invention relates to a kit comprising a pharmaceutical composition comprising a therapeutically effective amount of niclosamide and a pharmaceutically acceptable excipient, and instructions for administering the pharmaceutical composition. Another embodiment relates to a compound of formula I:

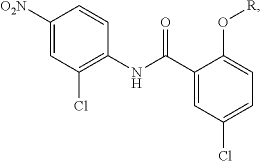

wherein R comprises at least one of CH₂)₂NH₃⁺ M; (CH₂)₂N⁺(CH₃)₃ M; (CH₂)₂SO₃⁻ M; (CH₂)₂PO₃⁻ M; CH₂CH₂(OCH₂CH₂)₃OH; or COCH₂(OCH₂CH₂)₃OH and M comprises a pharmaceutically acceptable salt.

In another embodiment, R comprises at least one of:

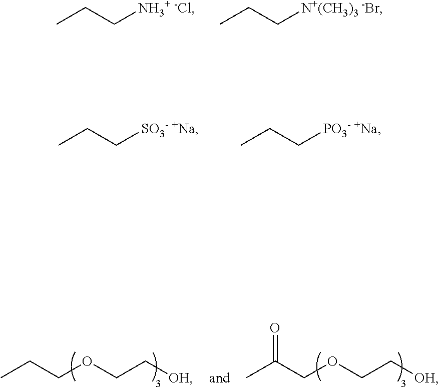

or mixtures thereof.

Another embodiment includes a compound comprising:

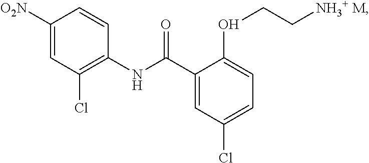

where M comprises a pharmaceutically acceptable salt. In a further embodiment, M comprises Cl⁻.

In another embodiment, the compound has a water solubility greater than about 1 mM.

Another embodiment of the invention relates to a medicament comprising the compound, pharmaceutically acceptable salt, or mixture of a compound described above. Some embodiments include the compound in a solid dosage unit suitable for oral administration. Some embodiments include the medicament in a therapeutically effective amount of the compound for the treatment of endometriosis in a subject in need of treatment.

Another embodiment of the invention relates to a kit comprising the medicament of claim 22. In some embodiments, the kit further comprises instructions for administering the medicament.

Another embodiment of the invention relates to a method of treating endometriosis comprising administering to a subject in need thereof a therapeutically effective amount of the compound described above. In some embodiments, the method is associated with reduction of endometrial lesions in the subject. In some embodiments, the method is associated with reduction of endometrial pain in the subject.

### III Examples

The following examples are provided to illustrate various aspects of the present disclosure, and should not be construed as limiting the disclosure only to these particularly disclosed embodiments.

The materials and methods used in the examples below are for illustrative purposes only, and are not intended to limit the practice of the present embodiments herein. Any materials and methods similar or equivalent to those described herein as would be apparent to one of ordinary skill I the art can be used in the practice or testing of the present embodiments.

### Example I: Mammalian Model of Endometriosis

An experimental animal endometriosis model was developed by obtaining endometrial fragments from one set of mice and implanting those fragments into another set of mice.

Mice were maintained in a vivarium at Southern Illinois University according to the NIH guidelines for the care and use of laboratory animals (Assurance A3078-01). Tg(UBC-GFP)30Scha (Strain of Origin: C57BL/6, aka B6-GFP, Jax #004353) and C57BL/6 (aka B6, Jax #000664) mice were obtained from Jackson Laboratory. The genotypes of B6-GFP mice were determined by PCR analysis of tail genomic DNA as previously described [Schaefer, 2001].

An experimental mouse model of endometriosis was established adopting procedures described previously with some modification [Han, 2012; Hirata, 2005; Kulak, 2011; Pelch, 2012; Zhao, 2014]. Female, eight week old B6-GFP mice were used as donor mice. These mice are transgenic mice whose cells endogenously express a green fluorescent protein (GFP) which is detectable under fluorescent light. When implanted to a GFP-negative recipient mouse, tissue implants from GFP-positive donor mice can be distinguished from the tissues of the recipient mice. Ectopic GFP-positive lesions were microscopically examined to evaluate the implants for accurate growth and progression.

Female, eight week old C57BL/6 mice were used as recipient mice. The donor mice and recipient mice have the same genetic background.

Briefly, the uterine horns were removed from donor mice during the diestrus stage of the reproductive cycle. Both horns were opened longitudinally and cut into a total of 4-tissue pieces (2 sets of each diameter punch) using 2-mm and 3-mm dermal biopsy punches (#15111-20 for 2-mm and #15111-30 for 3-mm, Ted Pella). Then, the uterine pieces were maintained in warmed DMEM/F12 medium (#10-090, Corning).

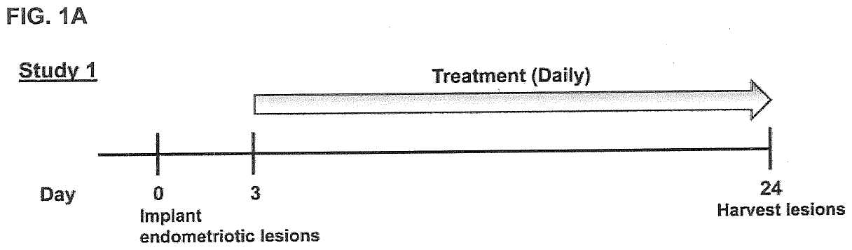

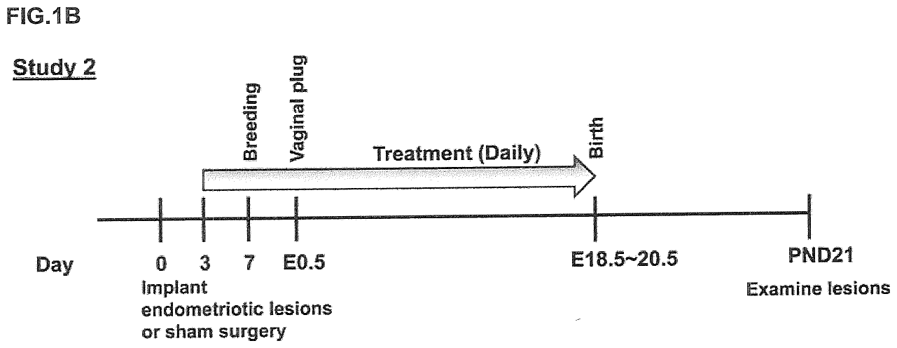

General outlines of further studies using niclosamide in the treatment of endometriosis are shown in FIGS. 1A-1B and described in detail below.

As shown in FIG. 1A, the recipient mice, B6 mice (8-weeks-old), were given endometriotic lesions. The recipient mice were selected during the diestrus stage and anesthetized. In each mouse, a longitudinal abdominal incision was made, and uterine pieces from donor mice were sutured to the right or left side of the peritoneal walls of each animal (implanting each 2-mm and 3-mm piece per side) using a 6-0 braided silk suture (# SUT-1073-11, Roboz). Then, the abdominal incision was closed with a 4-0 braided silk suture (# SUT-1073-31, Roboz). The mice were allowed to recover after surgery for three days, and then oral niclosamide was administered daily for 21 days.

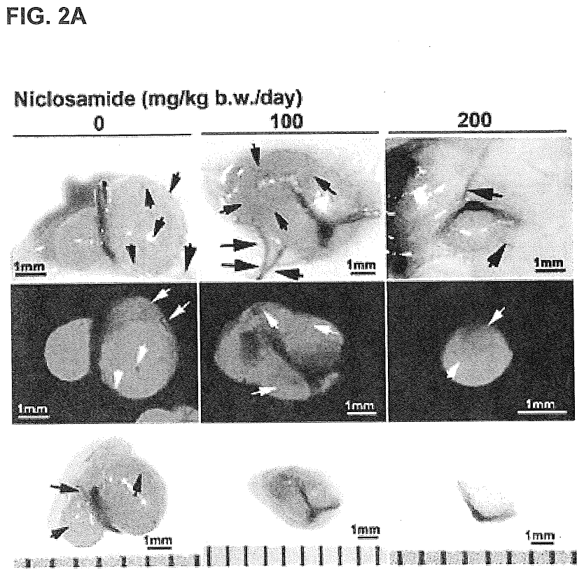

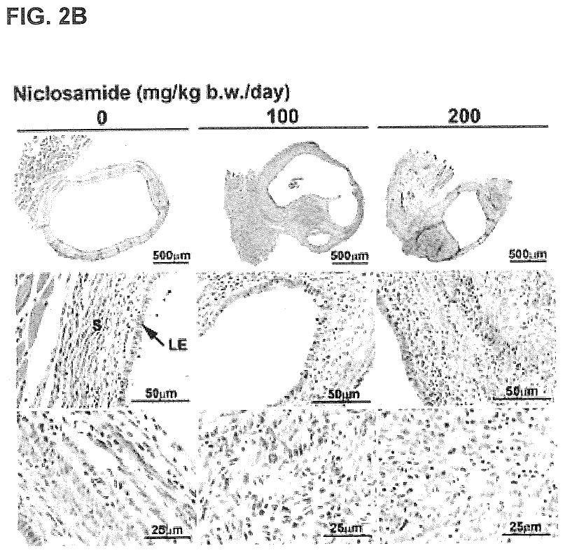

FIG. 2A shows microscopic images of endometriotic lesions examined after 3 weeks of treatment at doses of 0, 100 or 200 mg/kg b.w./day of niclosamide. The upper panels show sutured endometriotic lesions in situ, which have adhered to the peritoneal walls of recipient mice, and vascularization to the endometriotic lesions. The middle panels show the same sites examined under fluorescent light; the GFP-positive lesions are visible under fluorescent light, while the GFP-negative recipient tissues are not detectable under fluorescent light. The bottom panels show the endometriotic lesions after they were isolated from the recipient mice.

As seen in FIG. 2A, the endometriotic lesions adhered to the peritoneum of recipient mice, and obvious vascularization formed between the peritoneal wall and the endometriotic lesions (arrows in FIG. 2A). GFP-positive endometriotic lesions are clearly distinguishable from GFP-negative recipient tissues using fluorescence microscopy (middle panels in FIG. 2A).

Histological analysis confirmed that endometriotic lesions were attached to the peritoneal wall tissues, and intact epithelial and stromal cells of the endometriotic lesions were observed in control and niclosamide treated mice (FIG. 2B).

FIG. 2B shows histological sections of endometriotic lesions. The tissues were stained with hematoxylin and eosin. The upper panels show endometriotic lesions attached to peritoneal wall tissues. The middle and bottom panels show intact epithelial and stromal cells of the endometriotic lesions in control and niclosamide treated mice. After three weeks, the recipient mice displayed readily identifiable endometriotic lesions that originated from the donor mice, providing a mouse model of endometriosis for further studies.

### Example II: Oral Administration of Niclosamide Inhibits the Growth of Endometriotic Lesions

To investigate the effect of niclosamide on the growth and progression of endometriosis, endometriotic lesions were surgically implanted on the peritoneal walls of recipient mice, which were subsequently treated with orally administered niclosamide at 0, 100, or 200 mg/kg body weight, administered daily.

Uterine tissue pieces of B6-GFP mice (donor) were implanted into the peritoneal walls of recipient mice. As shown in FIG. 1A, endometriotic lesions were implanted into mice as described above. After 3 days of recovery after the transimplantation surgery, recipient mice (n=5-10) orally received niclosamide (# N3510, Sigma-Aldrich) at a dose of 0, 100 or 200 mg/kg of body weight (b.w.) per day for 3 weeks.

For oral administration, niclosamide was mixed in gelatin (Knox) with artificial flavors (Sweetener, Splenda®, and Berry Pomegranate, MiO®) and fed to the mice. The mice were trained to eat the flavored gelatin; after a few days of training, more than 95% of the mice ate their complete dosage of niclosamide (mixed in about 150 mm3 of gelatin) within 30 minutes. Mice that failed to eat all of their gelatin were eliminated from the studies.

After 3 weeks of treatment with niclosamide or control, all of the recipient mice appeared healthy with no obvious adverse effects including weight loss (data not shown). Estrous cyclicity was observed in all mice by vaginal cytology.

After 3 weeks of niclosamide treatment, the recipient mice in the diestrus stage were necropsied, and the endometriotic lesions were examined under a fluorescence stereo microscope (Leica) and collected for further analysis. The lesion volume was calculated according to the formula (L×W²), where L is length and W is width [Yoshioka, 2012; Satpathy, 2007].

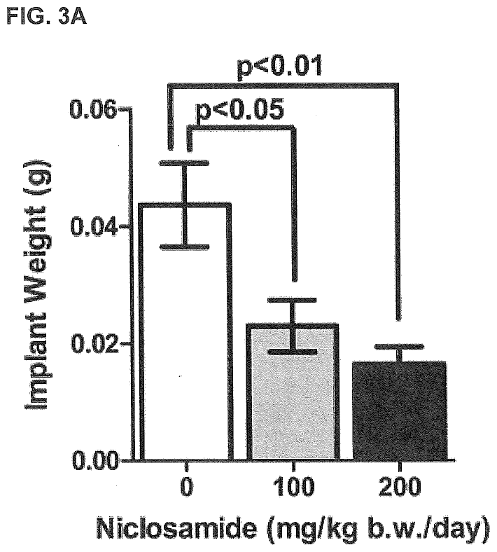

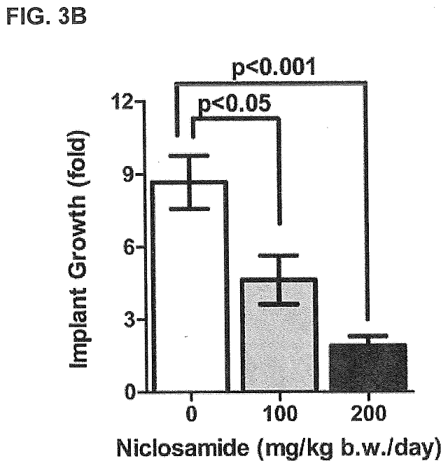

When the recipient mice were necropsied after 3 weeks of niclosamide treatment, a significant difference in the pattern of progression of endometriotic lesions was observed in niclosamide-treated mice. After three weeks of daily treatment with niclosamide at 0, 100, or 200 mg/kg of body weight, the endometriotic lesions was harvested from the recipient mice, and the weight and size of the endometriotic lesions was evaluated. The mean weights and increases in lesion volumes of the lesions obtained from niclosamide-treated mice are shown in FIGS. 3A-3B and show decreased tumor weight and growth with niclosamide treatment.

While the number of lesions did not differ between treatment groups, oral administration of niclosamide dose-dependently (50, 100, or 200 mg/kg b.w.) reduced the total weight of lesions 114.9±31.6 mg (n=7), 76.2±18.3 mg (n=9), or 44.9±7.43 mg (n=20), respectively, compared with controls (154.0±29.3 mg).

As shown in FIGS. 3A-3B, niclosamide treated mice that received a dose of 200 mg/kg b.w./day showed reduced lesion weight (0.016±0.003 g) and volume (7.4±0.8 mm3) compared to controls (lesion weight: 0.044±0.007 g, and lesion volume: 40.1±6.6 mm3). Niclosamide treated mice that received a dose of 100 mg/kg b.w./day also showed a significant reduction of lesion weight (0.023±0.004 g) compared to controls, whereas no significant difference was observed in lesion volume between controls and mice treated niclosamide with 100 mg/kg b.w./day (23.15±5.0 mm3).

### Example III: Oral Administration of Niclosamide Inhibits Cell Proliferation in Endometriotic Lesions without Affecting Hormone Signaling

To determine whether reduced lesion sizes after treatment with niclosamide resulted from alterations in cell proliferation, angiogenesis and/or apoptosis, the lesions were subjected to immunohistochemical analysis of proteins or other cellular components associated with those cellular processes.

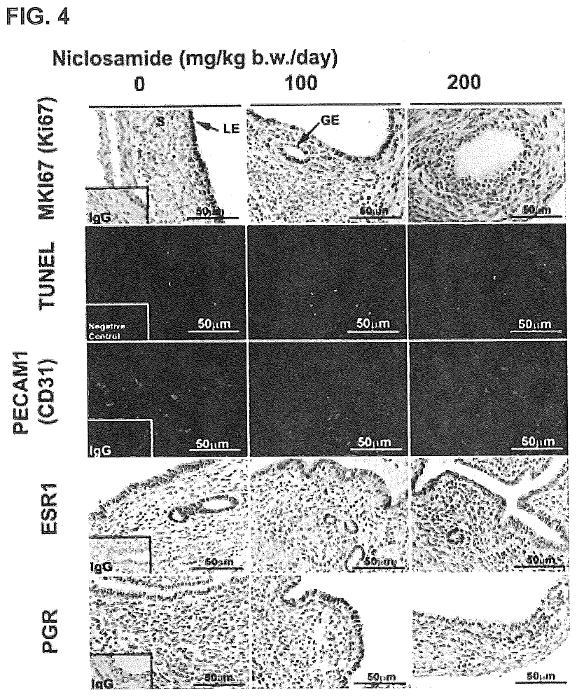

As shown in FIG. 4, endometriotic lesions were subjected to immunohistochemical analysis for the expression of MKI67 (Ki67) and PECAM1 (CD31), ESR1, and PGR, and apoptosis by TUNEL assay. Immunohistochemical or TUNEL analyses were semiquantitatively scored by counting of positively stained cells and/or total cells.

### Immunolocalization of Inflammatory Proteins in Uterine Tissues

The presence and location of different proteins in the tissues obtained from the recipient mice. Samples of uterine tissue were isolated from the recipient mice, fixed by standard histological methods, and embedded in paraffin. Thin sections (5 μm) of the paraffin-embedded tissues were screened with standard immunohistochemical methods using antibodies to identify the expression of in the uterine tissues.

Primary antibodies that recognized MKI67 (Ki67), PECAM1 (CD31), ESR1, PGR, p-CHUK (IKK), p-STAT3, NOS2 (iNOS) and PTGS2 (COX2), and Vectastain Elite ABC Kit (# PK-6101), Mouse on Mouse Basic Kit (# BMK-2202, Vector laboratories) or DyLight-conjugated secondary antibody (#711-516-152, Jackson ImmunoResearch Lab) were used to show the presence of the proteins in the tissues, which were examined under microscopes.

The primary antibodies used in these analyses were: anti-MKI67 (Ki67, 1:200 dilution, 550609, BD Biosciences), anti-PECAM1 (CD31, 1:100 dilution, ab28364, Abcam), anti-ESR1 (1:100 dilution, sc-542, Santa Cruz Biotechnology), anti-PGR (1:200 dilution, RB-9017-P0, Thermo Scientific), anti-p-CHUK (IKK, 1:150 dilution, 2697, Cell Signaling Technology), anti-p-STAT3 (1:50 dilution, 9145, Cell Signaling Technology), anti-NOS2 (iNOS, 1:50 dilution, 610333, BD Biosciences) and anti-PTGS2 (COX2, 1:50 dilution, RM-9121, Thermo Scientific).

### TUNEL Analysis of Uterine Tissues

The TUNEL assay is another immunohistological method used to show the amount of apoptosis, or programmed cell death, present in the uterine tissues. The TUNEL assay was performed on sections of uterine tissue according to manufacturer's instructions using ApopTag Fluorescein In Situ Apoptosis Detection Kit (# S7160, Millipore).

### Quantification of Cell-Specific Markers

The expression of MKI67, ESR1, PGR, p-CHUK, p-STAT3, NOS2 and PTGS2 proteins in uterine tissues, as determined by immunohistochemical analysis of uterine tissue sections, was semiquantitatively analyzed. Levels of apoptosis were analyzed by TUNEL assay and also semiquantitatively analyzed. The results of semiquantitatively analysis are shown in Tables 1 and 2. Histological sections were obtained from 4 different lesions. For each section, three different areas measuring 0.007 mm² were chosen. In each of those areas, the total number of epithelial or stromal cells was counted, as was the number of cells that tested positive for expressing MKI67 (a marker of cellular proliferation), steroid receptors ESR1 and PGR, and inflammatory signaling molecules p-CHUK, p-STAT3, NOS2 and PTGS2 proteins.

The expression of PECAM1, a marker of endothelial cells, was semiquantitatively analyzed using areas of 0.02 mm² (3 different areas counted from each section, with sections obtained from 4 different lesions), and semiquantitatively analyzed.

The TUNEL assay, which identifies cells undergoing apoptosis, was semiquantitatively analyzed using areas of 0.02 mm² (3 different areas counted from each section, with sections obtained from 4 different lesions), and semiquantitatively analyzed.

### Results

The majority of the cells, especially epithelial cells, expressed detectable amounts of MKI67 in the control lesions (FIG. 4). However, in agreement with the reduction of growth and progression of endometriotic lesions, fewer cells were MKI67 positive in the lesions of niclosamide treated mice. As shown in Table 1, niclosamide treatment at a dose of 200 mg/kg b.w./day significantly reduced epithelial cell proliferation, as shown by the expression of epithelial marker MKI67, to 37.7±7.2% compared with 61.6±5.9% of control lesions. There were no significant differences resulting from a dose of 100 mg/kg b.w./day niclosamide treatment (50.1±7.9%). No differences were observed in stromal cell proliferation, apoptosis, and PECAM1 staining in any endometriotic lesions. These results suggest that 200 mg/kg b.w./day niclosamide treatment resulted in decreased proliferation in the cells in the endometriotic lesions.

TABLE 1

| | | Niclosamide (mg/kg b.w./day) | | |
|---|---|---|---|---|
| | % | 0 | 100 | 200 |
| MKI67 (Ki67) | Epithelium | 61.6 ± 5.9 | 50.1 ± 7.9 | 37.7 ± 7.2* |
| | Stroma | 13.8 ± 1.4 | 10.9 ± 1.9 | 7.9 ± 1.9 |
| | | Niclosamide (mg/kg b.w./day) | | |
| Cell number | | 0 | 100 | 200 |
| TUNEL | | 8.7 ± 1.3 | 8.2 ± 1.3 | 7.8 ± 1.0 |
| PECAM1 (CD31) | | 38.3 ± 3.6 | 34.7 ± 3.6 | 32.7 ± 2.8 |
| | | Niclosamide (mg/kg b.w./day) | | |
| | % | 0 | 100 | 200 |
| ESR1 | Epithelium | 79.1 ± 3.7 | 96.6 ± 2.0 | 82.6 ± 5.6 |
| | Stroma | 45.6 ± 6.9 | 55.9 ± 5.5 | 37.7 ± 5.7 |
| PGR | Epithelium | 76.7 ± 7.0 | 60.4 ± 10.7 | 69.9 ± 12.2 |
| | Stroma | 47.2 ± 4.0 | 58.1 ± 5.2 | 50.1 ± 6.9 |

% of MKI67 positive cells/total cells/0.007 mm²
*p < 0.05 vs 0 mg/kg b.w./day of niclosamide
Cell number of TUNEL or PECAM1 positive cells/0.02 mm²
% of ESR1 or PGR positive cells/total cells/0.007 mm²

Furthermore, epithelial and stromal hormone receptors for estrogen and progesterone, ESR1 and PGR, were positively detected in the endometriotic lesions, and there were no differences between control and niclosamide treated mice (FIG. 4; Table 1), suggesting that niclosamide does not affect steroid hormone signaling. In turn, this suggests that the mechanism(s) by which niclosamide inhibited cell proliferation in endometrial cells operated independently of steroid hormone signaling.

### Example III: Oral Administration of Niclosamide Downregulates Inflammatory Signaling Related to STAT3 and NFκB Pathways

### Differential Expression of Proteins Involved in Inflammatory Signaling

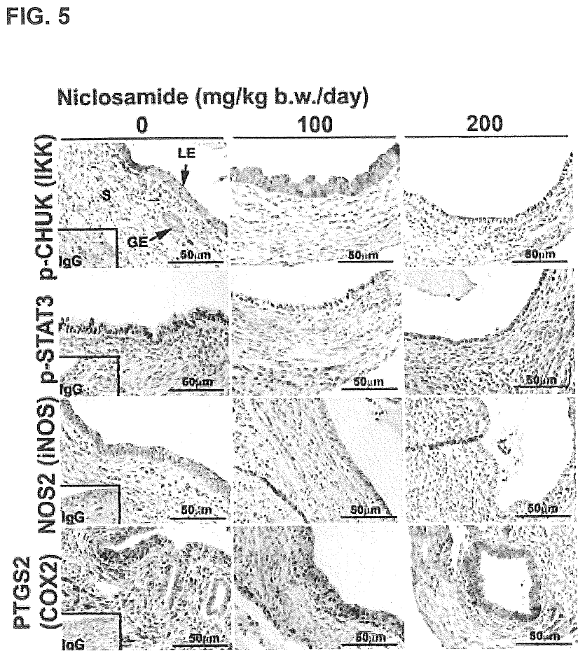

Because niclosamide was shown to target NFκB and STAT3 signaling in cancer cells, the activation of molecules in those signaling pathways were examined: CHUK, STAT3, and NFκB downstream molecule NOS2 (iNOS) using the immunohistochemical methods described above with primary antibodies that recognize p-CHUK (a portion on CHUK), p-STAT3 (a portion of STAT3), NOS2, or PTGS2 (FIG. 5). The expression of PTGS2 (COX2), an inflammatory molecule not activated by NFκB and STAT3 signaling, was examined with a primary antibody against PTGS2.

Immunohistochemical analysis revealed a reduction in the expression of STAT3 and iNOS proteins in endometriotic lesions following treatment with niclosamide. The expression of immunoreactive CHUK was significantly reduced in the epithelial and stromal endometriotic lesions treated with niclosamide at a dose of 200 mg/kg b.w./day. This dose of niclosamide treatment significantly decreased STAT3 activity and NOS2 in the epithelial cells of endometriotic lesions. However, PTGS2 was not affected by niclosamide treatment. Semiquantitative analysis was performed on the expression of p-CHUK, p-STAT3, NOS2, and PTGS2 (Table 2), also showed that niclosamide treatment at 200 mg/kg b.w./day resulted in a statistically significant reduction in the expression of p-CHUK, p-STAT3, and NOS2, but not PTGS2, proteins in the endometriotic lesions. These results suggest that niclosamide can treat endometriosis by targeting inflammatory molecules controlled by the NFκB and STAT3 signaling pathways, independent of prostaglandin pathways and steroid hormone signaling pathways.

TABLE 2

| | | Niclosamide (mg/kg b.w./day) | | |
|---|---|---|---|---|
| | % | 0 | 100 | 200 |
| P-CHUK (IKK) | Epithelium | 97.9 ± 2.1 | 92.3 ± 3.8 | 76.7 ± 9.2* |
| | Stroma | 83.4 ± 4.7 | 86.3 ± 4.0 | 56.2 ± 8.3** |
| p-STAT3 | Epithelium | 54.1 ± 3.9 | 59.0 ± 5.4 | 26.4 ± 3.8*** |
| | Stroma | 11.5 ± 4.4 | 6.9 ± 1.9 | 4.1 ± 1.0 |
| NOS2 (iNOS) | Epithelium | 99.5 ± 0.4 | 99.5 ± 0.5 | 86.2 ± 5.9* |
| | Stroma | 75.2 ± 4.6 | 57.8 ± 4.3 | 61.9 ± 5.7 |
| PTGS2 (COX2) | Epithelium | 98.4 ± 0.5 | 98.2 ± 0.5 | 98.7 ± 0.7 |
| | Stroma | 20.1 ± 2.2 | 20.3 ± 3.3 | 22.8 ± 2.0 |

% of p-CHUK, p-STAT3, NOS2 or PTGS2 positive cells/total cells/0.007 mm²
*p < 0.05, **p < 0.01 or ***p < 0.001 vs 0 mg/kg b.w./day of niclosamide

### Differential Expression of Genes Involved in Inflammatory Signaling

Because these results suggested that reduction of growth and progression of endometriotic lesions by niclosamide are through inhibition of inflammatory mechanisms, RNA sequencing was performed to identify genes whose expression is regulated by niclosamide in endometriotic lesions.

The endometriotic lesions were screened by RNA sequencing for the expression of a variety of genes of interest. Total RNA was isolated from the lesions using the RNeasy mini kit (#74104, Qiagen). RNA quality was assessed, and RNA-sequencing (RNA-seq) were performed at the Functional Genomics Core Facility of University of Illinois. Briefly, the stranded RNA-seq libraries were prepared with IIlumina's TruSeq Stranded RNA Sample Prep kit (# RS-122-2201, Illumina). The libraries were pooled in equimolar concentration, and the pool was quantitated by qPCR and sequenced on one lane for 101 cycles on a HiSeq2500 (Illumina) using a HiSeq SBS sequencing kit (# FC-401-4002, Illumina). Fastq files were generated and demultiplexed with the bcl2fastq v1.8.4 Conversion Software (Illumina).

Quantitative PCR (qPCR) was performed as described previously [Reardon, 2012]. The 5′-3′ sequences of the primer pairs (forward and reverse primer pairs) used to determine the presence of different genes are shown in Table 3. For each gene, the name of the gene, the accession number of the gene, the 5′-3′ sequence of the paired primers, and the length of the amplicon obtained is provided in Table 3.

TABLE 3

| Gene | Accession No. | Dir. | Primer Sequence | SEQ ID NO. |
|---|---|---|---|---|
| Adipoq | NM_009605.4 | For | CTCCACCCAAGGGAACTT | SEQ ID NO: 1 |
| | | | GT | |
| | | Rev | TAGGACCAAGAAGACCT | SEQ ID NO: 2 |
| | | | GCATC | |
| Ano4 | NM_001277188.1 | For | CCCAAAGAGCGATGTGG | SEQ ID NO: 3 |
| | | | ACT | |
| | | Rev | CTTCTAGCCTGCTGGTGT | SEQ ID NO: 4 |
| | | | CC | |
| Ccl20 | NM_001159738.1 | For | CGACTGTTGCCTCTCGTA | SEQ ID NO: 5 |
| | | | CA | |
| | | Rev | GAGGAGGTTCACAGCCC | SEQ ID NO: 6 |
| | | | TTT | |
| Ccl28 | NM_020279.3 | For | TGTGTGTGGCTTTTCAAA | SEQ ID NO: 7 |
| | | | CCT | |
| | | Rev | GTACGATTGTGCGGGCT | SEQ ID NO: 8 |
| | | | GAT | |
| Cxcl9 | NM_008599.4 | For | AGTTCGAGGAACCCTAGT | SEQ ID NO: 9 |
| | | | GA | |
| | | Rev | TTGTAGTGGATCGTGCCT | SEQ ID NO: 10 |
| | | | CG | |
| Cxcl14 | NM_019568.2 | For | AAAGTACCCACACTGCG | SEQ ID NO: 11 |
| | | | AGG | |
| | | Rev | CTTCGTAGACCCTGCGCT | SEQ ID NO: 12 |
| | | | TC | |
| Cxcr2 | NM_009909.3 | For | ATCTTCGCTGTCGTCCTT | SEQ ID NO: 13 |
| | | Rev | GAAGCCAAGAATCTCCG | SEQ ID NO: 14 |
| | | | TAG | |
| Cxcr6 | NM_030712.4 | For | CTGGGGTTCTTCCTGCCA | SEQ ID NO: 15 |
| | | | TT | |
| | | Rev | ATGGCAAGGATTGAAGG | SEQ ID NO: 16 |
| | | | GTGT | |
| Dkk2 | NM_020265.4 | For | CAGTCACTGAGAGCATC | SEQ ID NO: 17 |
| | | | CTCA | |
| | | Rev | CCTGATGGAGCACTGGTT | SEQ ID NO: 18 |
| | | | TGC | |
| Fzd10 | NM_175284.3 | For | ACAACCCAGGCAAGTTC | SEQ ID NO: 19 |
| | | | CACCA | |
| | | Rev | GCCAGCCAGACCACAGC | SEQ ID NO: 20 |
| | | | G | |
| Gja5 | NM_001271628.1 | For | CCAGAGCCTGAAGAAGC | SEQ ID NO: 21 |
| | | | CAA | |
| | | Rev | GAGCCTTCCACGTTTAGA | SEQ ID NO: 22 |
| | | | GC | |
| Id4 | NM_031166.2 | For | AGGGTGACAGCATTCTCT | SEQ ID NO: 23 |
| | | | GC | |
| | | Rev | CCGGTGGCTTGTTTCTCT | SEQ ID NO: 24 |
| | | | TA | |
| Ifng | NM_008337.4 | For | GGCAAAAGGATGGTGAC | SEQ ID NO: 25 |
| | | | ATGAA | |
| | | Rev | TTTTCGCCTTGCTGTTGCT | SEQ ID NO: 26 |
| | | | G | |
| Lep | NM_008493.3 | For | GACATTTCACACACGCAG | SEQ ID NO: 27 |
| | | | TCG | |
| | | Rev | ACATTTTGGGAAGGCAG | SEQ ID NO: 28 |
| | | | GCT | |
| Lpo | NM_080420.2 | For | ATGCAGTGGGGTCAAAT | SEQ ID NO: 29 |
| | | | CGT | |
| | | Rev | TGTTCGTCACACTGGGCT | SEQ ID NO: 30 |
| | | | TT | |
| Mmp7 | NM_010810.4 | For | GGCTTCGCAAGGAGAGA | SEQ ID NO: 31 |
| | | | TCA | |
| | | Rev | GAATGCCTGCAATGTCGT | SEQ ID NO: 32 |
| | | | CC | |
| Mmp9 | NM_013599.4 | For | ACACGACATCTTCCAGTA | SEQ ID NO: 33 |
| | | Rev | CACCTTGTTCACCTCATT | SEQ ID NO: 34 |
| Mmp17 | NM_011846.4 | For | CTGGCGCTATGATGACCA | SEQ ID NO: 35 |
| | | | CA | |
| | | Rev | GAAATAGGATGCACCAT | SEQ ID NO: 36 |
| | | | CAGACC | |
| Mrvi1 | NM_010826.5 | For | CCCCACATTCCTGAGGAT | SEQ ID NO: 37 |
| | | | GAG | |
| | | Rev | GGGCTGGGAAATGGTCC | SEQ ID NO: 38 |
| | | | TG | |
| Muc13 | NM_010739.2 | For | TCGTCTCTGCGAGGTCAA | SEQ ID NO: 39 |
| | | | AG | |
| | | Rev | TGCTGTTGTCCGCTCCTA | SEQ ID NO: 40 |
| | | | AG | |
| Nkd1 | NM_027280.3 | For | ACGCATGGCTTAGGACG | SEQ ID NO: 41 |
| | | | CTC | |
| | | Rev | GTCACCCACGAGTTCCC | SEQ ID NO: 42 |
| | | | GAG | |
| Ptprz1 | NM_001081306.1 | For | CTGTCTAGTGGTTCTTGT | SEQ ID NO: 43 |
| | | | TGGT | |
| | | Rev | GGGTGTTGGTGGTGTAG | SEQ ID NO: 44 |
| | | | ATATT | |
| Retn | NM_001204959.1 | For | GCTCGTGGGACATTCGTG | SEQ ID NO: 45 |
| | | | A | |
| | | Rev | ACCACATCCAGCCTGTTT | SEQ ID NO: 46 |
| | | | TGTT | |
| Ror2 | NM_013846.3 | For | CACCAGAGTGGCCCTCG | SEQ ID NO: 47 |
| | | | AA | |
| | | Rev | ATCTTCCACTTCACCTGC | SEQ ID NO: 48 |
| | | | CGTC | |
| Sprr2b | NM_011469.3 | For | GAACAATGTCTTACCACC | SEQ ID NO: 49 |
| | | | AGCA | |
| | | Rev | AGTCCTGATGACTGGGA | SEQ ID NO: 50 |
| | | | AACC | |
| Trpc3 | NM_019510.2 | For | ACCTCTTCACACAGTCTA | SEQ ID NO: 51 |
| | | | ACTCG | |
| | | Rev | TCAGTTCACCTTCATTCA | SEQ ID NO: 52 |
| | | | CCTCA | |
| Wnt7a | NM_009527.3 | For | GGCTCCCAGACAGCGGG | SEQ ID NO: 53 |
| | | | CAA | |
| | | Rev | CGGAACTGAAACTGACA | SEQ ID NO: 54 |
| | | | C | |

Methods involving conventional molecular biology techniques generally known in the art are described in detail in methodology treatises such as Molecular Cloning: A Laboratory Manual, 3rd ed., vol. 1-3, ed. Sambrook et al., Cold Spring Harbor Laboratory Press, Cold Spring Harbor, N.Y., 2001; and Current Protocols in Molecular Biology, ed. Ausubel et al., Greene Publishing and Wiley-Interscience, New York, 1992 (with periodic updates).

All experimental data were subjected to one-way ANOVA tests and the differences between individual means were tested by a Tukey multiple-range test using Prism 5.0 (GraphPad). qPCR data were tested by t-test using Prism 5.0. Tests of significance were performed using the appropriate error terms according to the expectation of the mean squares for error. A P-value of 0.05 or less was considered significant. Data are presented as mean with standard error of the mean (SEM).

Because a dose of 200 mg/kg b.w./day was more effective at reducing the size of lesions, RNA sequencing was performed on lesions obtained from recipient mice treated with control or a 200 mg/kg b.w./day niclosamide (n=3 each treatment) for 3 weeks, and the results were compared. A total of 15,220 genes (>1 count per million reads) were identified by RNA-sequencing. While a total of 951 genes were less than 0.05 of raw p-values, only a total of 199 genes were indicated less than 0.1 of false discovery rate (FDR). Therefore, differentially expressed genes (199 total genes, FDR P<0.1) were classified by functional annotation using DAVID analysis [Dennis, 2003; Huang da, 2009] and IPA program (Ingenuity Systems).

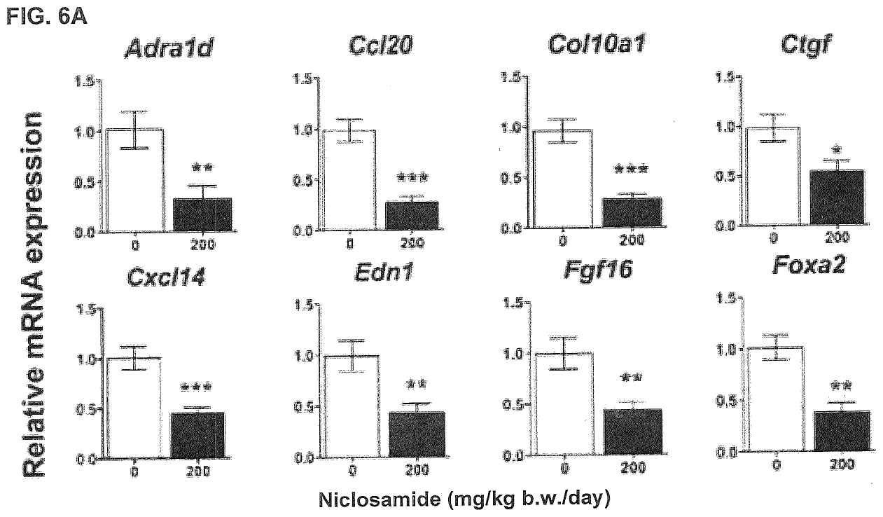

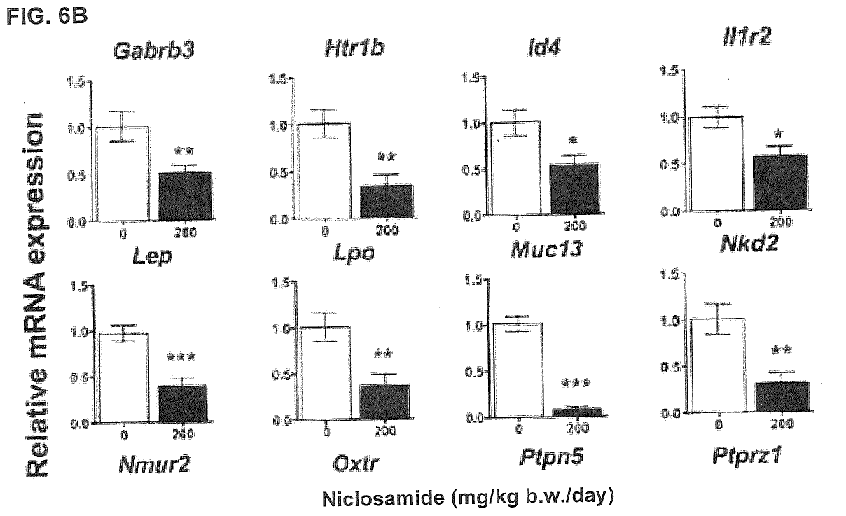

These 199 genes were categorized into groups of cell-to-cell signaling, extracellular matrix, inflammatory response, immune cell trafficking and cellular movement (Table 4). Differentially expressed transcripts were further confirmed by qPCR (n=6-8), as shown in FIGS. 6A-6C. These results highlighted that genes related to inflammation (Cxcl14, Lep, Lpo, Muc13, Trpc3 and Ptprz1) were significantly decreased in the endometriotic lesions after niclosamide treatment (FIGS. 6A-6C). Additionally, Id4 and Wnt7a were also reduced in treated lesions (FIGS. 6B-6C). Treatment of niclosamide decreased cytokine and chemokine levels including: II1b, Cxcr2, Csf3r, II1rn, Tnf, II10, and Osm, but increased Ccl17 in the endometriotic lesions.

TABLE 4

| | Gene | | |
|---|---|---|---|
| Bio Functions | count | p-value | Functions Annotation |
| Cell-to-cell signaling | 51 | 2.10E−11 | migration, chemotaxis and |
| | | | recruitment of cells |
| Extracellular matrix | 23 | 3.90E−08 | structural support for cells |
| Inflammatory response | 17 | 8.30E−07 | activation and chemotaxis |
| | | | of cells |
| Immune cell trafficking | 18 | 1.40E−04 | adhesion and migration |
| | | | of cells |
| Cellular movement | 12 | 3.50E−04 | activation of cells |

These studies show that niclosamide reduces the size of endometriotic lesions in a mouse model of endometriosis by targeting inflammatory responses, at the levels of both gene and protein expression.

### Example IV: Niclosamide Treatment does not Interfere with Reproductive Function

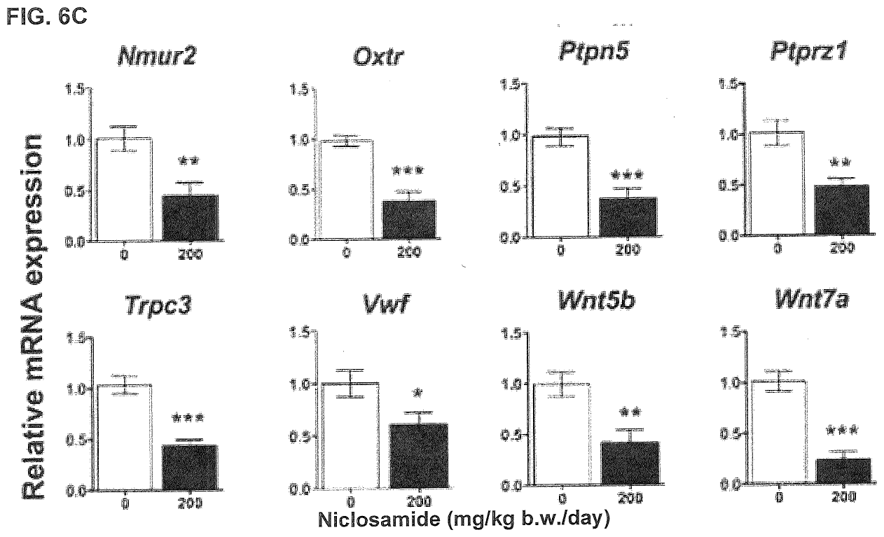

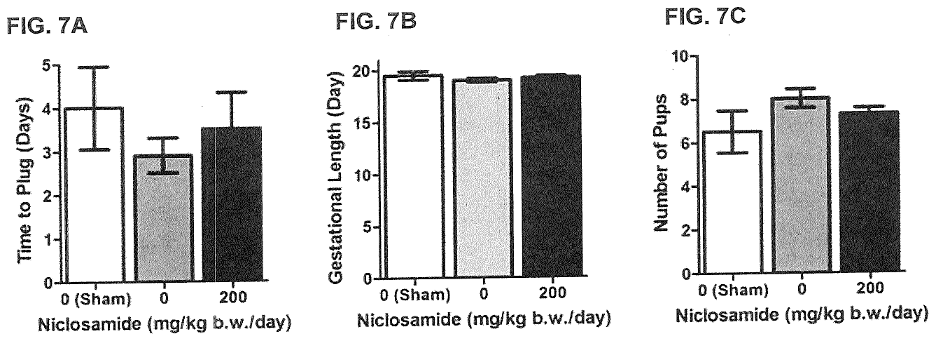

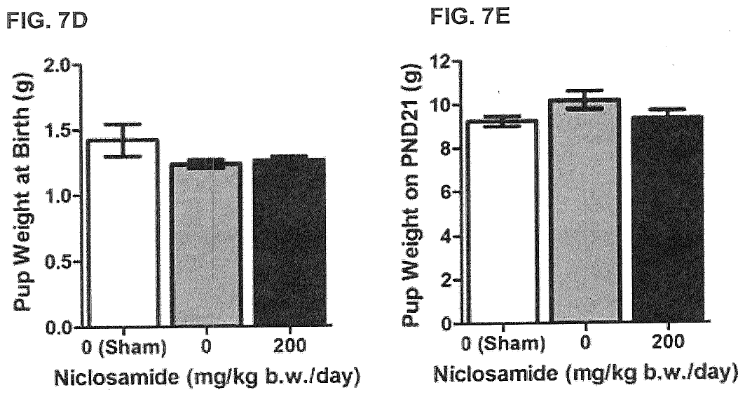

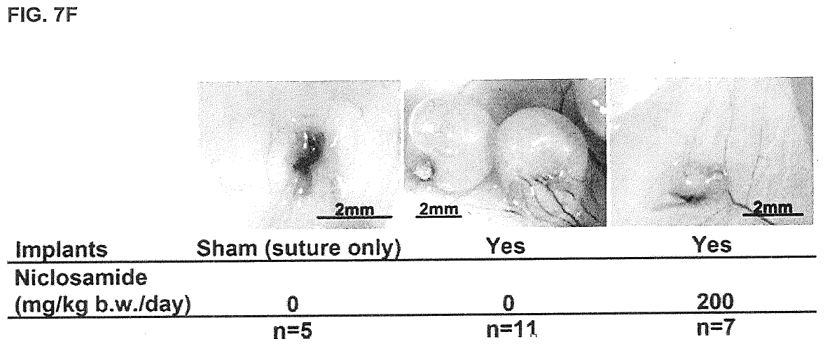

Currently, the most widely used drugs (GnRH agonists and progestins) suppress and/or disrupt normal ovarian function. Therefore, the effect of oral niclosamide treatment on reproductive function in mice was determined (FIGS. 7A-7G).

As shown in FIG. 1B, female recipient mice received endometriotic lesions (n=7-11) as described above or were subjected to sham surgery (n=5). The sham surgery was performed following the same steps as the endometriosis surgery except that no donor tissues were implanted onto the peritoneal walls of those mice; only sutures were applied to the peritoneal walls.

After 3 days of surgical recovery, both groups of recipient mice started receiving niclosamide administered orally at a dose of 0 or 200 mg/kg b.w./day. After 4 days of daily oral niclosamide treatment, the female recipient mice were mated with wild-type B6 male mice. The recipient mice continued receiving oral daily niclosamide treatments until mouse pups were born, after which niclosamide treatment was discontinued. Three weeks after the pups were born, on postnatal day (PND) 21, the recipient mice were necropsied and the pups were weaned.

For each mated mouse, the following parameters were measured and recorded: the time of the formation of the vaginal plug after mating (days); the length of time that the mated mouse gestated or was pregnant (days); the number of pups born to each mated mouse; and the size and volume of the endometriotic lesions harvested from the mice after necropsy. Tissues were harvested from the mice, including endometriotic lesions and peritoneal walls to analyze the expression of proteins and genes of interest.

For each pup born to a mated mouse, its weight (g) at birth and on PND 21 was measured.

No significant differences were detected after the female mice were bred (FIGS. 7A-7B). Most of the mice had vaginal plugs 1 to 4 days after breeding. No significant differences of time to receive vaginal plug were observed between the groups, suggesting that niclosamide does not affect ovarian function. In fact, all of the recipient mice exposed to niclosamide became pregnant and gave birth.

Niclosamide treatment did not cause any differences in gestational length, number of pups, and weight of pups at birth and on PND21 (FIGS. 7B-7E). These results suggest that niclosamide does not disturb any uterine functions including conception, implantation, duration of pregnancy, fetal growth, and parturition. These results also suggest that niclosamide treatment does not adversely affect the pups in utero.

When the endometriotic lesions of the recipient mice were examined on PND 21, the treatment of niclosamide maintained the reduction of lesion sizes even though they were examined 3 weeks after the final dosage of niclosamide was administered (FIGS. 7B-7C).

These niclosamide toxicity studies demonstrate that the treatment of niclosamide had no observable impact on reproductive function in female mice.

Acute toxicity in mice is reported as LD50=>1500 mg/kg b.w. Thus, the dosages (maximum 200 mg/kg b.w.) used in our study, that were effective for lesion reduction, were much lower than the reported LD50 of acute toxicity in mice. Indeed, daily administration of niclosamide at a dose of 200 mg/kg b.w. for three in Study 1 (FIG. 1A) to four weeks (average) in Study 2 (FIG. 1B) did not cause any adverse reactions, such as weight loss or changes in behavior in the recipient mice.

### Example V: Niclosamide Derivatives

Niclosamide is also known as 5-chloro-N-(2-chloro-4-nitrophenyl)-2-hydroxybenzamide and is a chlorinated salicylanilide. Niclosamide's limitations as a drug include poor water solubility, minimal oral bioavailability (10%), and low plasma half-life (7h) in rats [Chang, 2006]. Although there has been increased interest in niclosamide's action against key pathological pathways as an anti-cancer drug; low solubility, low bioavailability and poor pharmacokinetic profile result in variation of its anti-cancer efficacy when it is used in clinical trials.

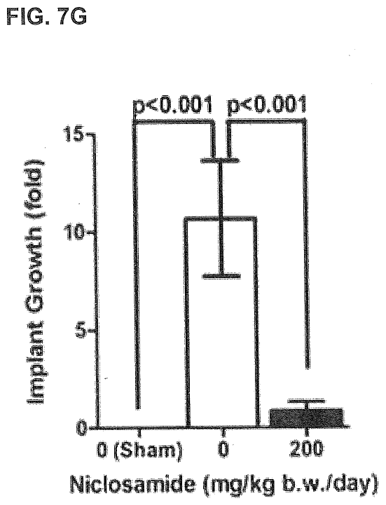

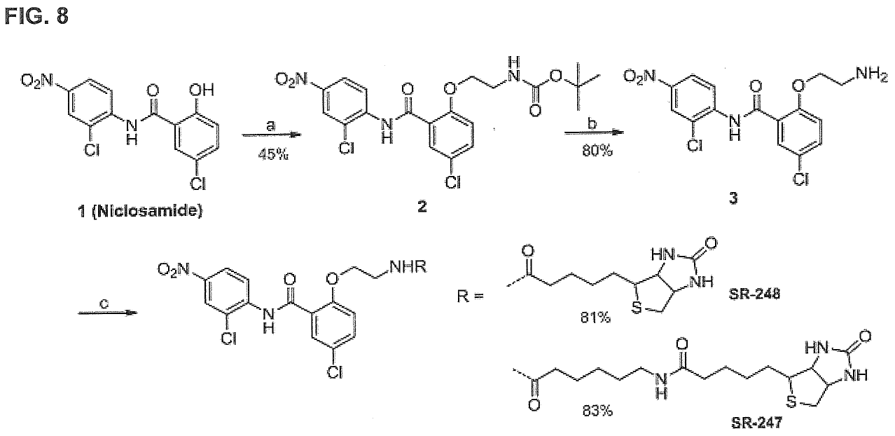

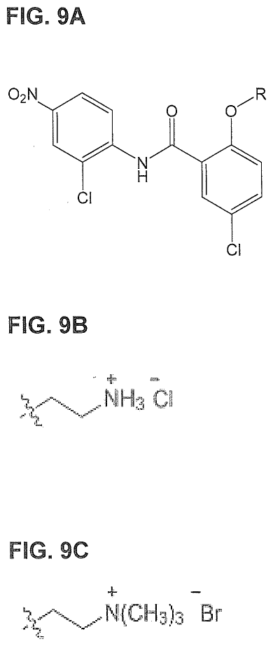

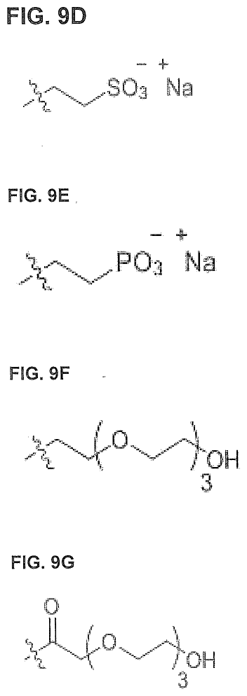

Niclosamide was modified to yield biotinylated versions of the molecule which can be used to identify molecules or structures that bind niclosamide (FIG. 8). Niclosamide can also be modified to yield derivatives suitable for therapeutic use in place of niclosamide (FIGS. 9A-9G).

Niclosamide derivatives have been synthesized or designed that incorporate modifications to niclosamide in the following Formula I:

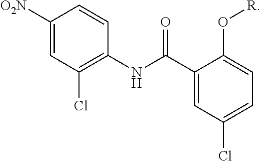

### Biotinylated Niclosamide Derivatives

The invention includes derivatives of niclosamide modified to include biotin conjugated to the molecule, in the procedures outlined in FIG. 8. Niclosamide was reacted with N-boc-aminoethanol, triphenylphosphine, diisopropyl azodicarboxylate, and tetrahydrofuran; then reacted with trifluoroacetic acid and dichloromethane and tetrahydrofuran; and then reacted with trimethylamine (Et₃N), dimethylformamide, and either EZ-Link-NHS-Biotin (to yield SR-248) or EZ-Link-Sulfo-NHS-LC-Biotin (to yield SR-247).

Niclosamide was thus modified to obtain two derivatives (SR-247 and SR-248) that include biotin conjugated to formula I. for use in identifying niclosamide's direct targets, especially in cells, tissues, and individual patients or subjects. The derivatives named SR-247 and SR-248 provide a biotin-conjugated derivative of niclosamide for screening or identifying targets of niclosamide, for example in organisms, cells, tissues, and other samples.

### Therapeutic Niclosamide Derivatives

The methods of the invention can be practiced by administering to an individual in need thereof an effective amount of a compound derived from niclosamide. Specifically, as shown in FIG. 9A

by the administration of an effective amount of a compound derived from formula I:

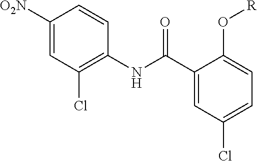

wherein R can be (CH₂)₂NH₂; (CH₂)₂N(CH₃)₂CH₃; (CH₂)₂SO₄; (CH₂)₂PO₄; CH₂CH₂(OCH₂CH₂)₂OCH₂CH₃; or COCH₂(OCH₂CH₂)₂OCH₂CH₃; or a pharmaceutically acceptable salt thereof, and mixtures thereof.

The compound can include formula I wherein R is (CH₂)₂NH₃⁺Cl⁻; (CH₂)₂N⁺(CH₃)₃ Br⁻; (CH₂)₂SO₃⁻Na⁺; (CH₂)₂PO₃⁻Na⁺; CH₂CH₂(OCH₂CH₂)₃OH; or COCH₂(OCH₂CH₂)₃OH and mixtures thereof. Niclosamide derivatives can be synthesized to comprise the structure illustrated in FIG. 9A, where the R group comprises a specific species listed in FIGS. 9B-9G. Where FIGS. 9B-9G recite a salt, that salt can be substituted with a pharmaceutically acceptable salt.

In one embodiment, niclosamide was modified by replacing the phenolic hydroxyl group of niclosamide with a free amine (FIG. 8, form 2 converted to form 3). Form 3 of FIG. 8 was further modified by acidification of the O-ethylamino group with HCl, to provide the protonated salt form described by FIG. 9A wherein R comprises the structure shown by FIG. 9B. This derivative displayed significantly improved water solubility (˜2 mM), which is approximately 100 times higher than niclosamide (<20 μM [ORGANIZATION WH, 2002]).

The improved solubility suggests that this derivative, or other water-soluble analogs, can be useful drug candidates in lieu of the parental niclosamide in a clinical setting.

Other derivatives of Formula I can have improved water solubility greater than about 1 mM, greater than about 500 μM, greater than about 100 μM, greater than about 50 μM, greater than about 20 μM, or greater than about 1 μM.

Other derivatives of niclosamide can be synthesized, designed for improved solubility, bioavailability, and/or efficacy in the treatment of endometriosis. These derivatives can also be suitable for use of the known use of niclosamide as an antihelminth agent in the treatment of tapeworm infections. These derivatives can also suitable for use in the treatment of various cancers.

The pharmaceutically acceptable salts of formula I compounds can have enhanced solubility characteristics compared to the niclosamide molecule from which they are derived, and thus can be more amenable to formulation as liquids or emulsions.

Thus, a contemplated compound or its pharmaceutically acceptable salt can optionally be present in one or more forms. Illustratively, the compound or its salt can be in the form of an individual enantiomer or diastereoisomer. A contemplated compound or its salt can also be present in the form of a mixture of stereoisomers. A contemplated compound or salt can also be present in the form of a racemic mixture.

Once prepared, the free base, free acid, or salt form of formula I compounds can be administered to an individual in need of treatment for the methods herein described or for other known applications for therapeutic administration of niclosamide.

### Pharmaceutical Compositions

Niclosamide or a niclosamide derivative or pharmaceutically acceptable salt thereof can be used in the manufacture of a medicament (pharmaceutical composition) that is useful for inhibiting growth of endometrial cells outside of the uterus of a subject as discussed herein in a mammal, as well as in mammalian cells and mammalian cell preparations. Niclosamide is also used as an antihelminth agent and as an anti-cancer agent.

Niclosamide or a niclosamide derivative or pharmaceutically acceptable salt thereof can be administered as a pharmaceutically acceptable preparation. Preparations can be administered in accordance with the invention in pharmaceutically acceptable compositions that can optionally comprise pharmaceutically acceptable salts, buffering agents, preservatives and excipients

A mammal in need of treatment and to which a pharmaceutical composition containing a contemplated niclosamide compound is administered can be a primate such as a human, an ape such as a chimpanzee or gorilla, a monkey such as a cynomolgus monkey or a macaque, a laboratory animal such as a rat, mouse or rabbit, a companion animal such as a dog, cat, horse, or a food animal such as a cow or steer, sheep, lamb, pig, goat, llama or the like.

A contemplated pharmaceutical composition can be administered orally (perorally), parenterally, by inhalation spray in a formulation containing conventional nontoxic pharmaceutically acceptable carriers, adjuvants, and vehicles as desired. Formulation of drugs is discussed in, for example, Hoover, John E., Remington's Pharmaceutical Sciences, Mack Publishing Co., Easton, Pa.; 1975 and Liberman, H. A. and Lachman, L., Eds., Pharmaceutical Dosage Forms, Marcel Decker, New York, N.Y., 1980.

Solid dosage forms for oral administration can include capsules, tablets, pills, powders, and granules. Compositions suitable for oral administration include capsules, cachets, tablets, syrups, elixirs or lozenges. The niclosamide can also be incorporated into food and administered orally. In such solid dosage forms, a contemplated compound is ordinarily combined with one or more excipients appropriate to the indicated route of administration. If administered per os, the compounds can be admixed with gelatin, lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation as can be provided in a dispersion of active compound in hydroxypropylmethyl cellulose. In the case of capsules, tablets, and pills, the dosage forms can also comprise buffering agents such as sodium citrate, magnesium or calcium carbonate or bicarbonate. Tablets, capsules and pills can additionally be prepared with enteric coatings.

A contemplated pharmaceutical composition contains an amount of niclosamide or a contemplated niclosamide derivative or a pharmaceutically acceptable salt thereof (or mixtures thereof) dissolved or dispersed in a physiologically tolerable carrier. Such a composition can be administered to mammalian cells in vitro as in a cell culture, or in vivo as in a living, host mammal in need.

Preferably, the pharmaceutical composition is in unit dosage form. In such form, the composition is divided into unit doses containing appropriate quantities of the niclosamide or niclosamide derivative or thereof as active agent. The unit dosage form can be a packaged preparation, the package containing discrete quantities of the preparation, for example, in vials or blister packs.

The pharmaceutical composition can be part of a kit, which can further include instructions for administering the pharmaceutical composition.

Several useful contemplated niclosamide compounds can typically be used in the form of a pharmaceutically acceptable acid addition salt derived from an inorganic or organic acid. The reader is directed to Berge, J. Pharm. Sci. 68(1):1-19 (1977) for lists of commonly used pharmaceutically acceptable acids and bases that form pharmaceutically acceptable salts with pharmaceutical compounds.

In some cases, the salts can also be used as an aid in the isolation, purification or resolution of the compounds of this invention. In such uses, the acid used and the salt prepared need not be pharmaceutically acceptable.

### Administration of Pharmaceutical Compositions

The mode of administration selected will depend on the acuteness and severity of the condition being treated, and the dosage required. Any mode of administration that produces desired therapeutic effect without unacceptable adverse effects is relevant in practicing the invention. Such modes of administration can include oral, rectal, topical, transdermal, sublingual, intramuscular, parenteral, intravenous, intracavity, vaginal, and adhesive matrix to be used during surgery. Various approaches for formulating compositions for use in accordance with the invention are described in the Handbook of Pharmaceutical Excipients, Third Edition, American Pharmaceutical Association, USA and Pharmaceutical Press UK (2000), and Pharmaceutics—The Science of Dosage Form Design, Churchill Livingston (1988).

Preferably the niclosamide or niclosamide derivative is administered in the form of a preparation which includes one or more of niclosamide or niclosamide derivatives as the active ingredient.

The niclosamide can be administered at a dose (e.g. an oral dose to a human patient) of between 50 milligrams kilogram of body weight per day and 250 mg/day, preferably between 100 mg/kg b.w./day and 200 mg/kg b.w./day, more preferably about 200 mg/kg b.w./day; suitable doses within this range depend on the niclosamide to be used, as is readily apparent to those skilled in the art.

The niclosamide can be administered as, for example, a single daily dose (of for example, between 50 milligrams kilogram of body weight per day and 250 mg/day, preferably between 100 mg/kg b.w./day and 200 mg/kg b.w./day, more preferably about 200 mg/kg b.w./day); or the daily dose can be divided into two or more sub-doses to be taken at different times over a 24 hour period. The niclosamide can be administered as a daily dose at the levels above, or as equivalent doses e.g. per week, twice a week, or every two days.

The niclosamide can be administered for long periods of time (e.g. 1 to 3 weeks); from 1 day to 1 month). The administration can be continuous at the daily or weekly dose, or can be interrupted by one or more interruptions of, for example, a number (e.g. 1-3) of weeks or a number (e.g. 1 to 3) of months. The niclosamide can be administered as long as symptoms (such as pain) continue.

In one embodiment, niclosamide can be used as the only medical treatment for endometriosis. In other words, the niclosamide can be used in the absence of other medical or surgical treatments [for example, in the absence of progestins].

In a further embodiment, administration of niclosamide can be combined with other medical or surgical treatments for endometriosis; for example, NSAIDs and/or hormonal treatments (progestins, GnRH agonists and antagonists,). In a further embodiment, surgical treatment or medical treatment can be used prior, during or after treatment with niclosamide.

### OTHER EMBODIMENTS

Any improvement can be made in part or all of the composition, kit, and method steps. All references, including publications, patent applications, and patents, cited herein are hereby incorporated by reference. The use of any and all examples, or exemplary language (e.g., “such as”) provided herein, is intended to illuminate the invention and does not pose a limitation on the scope of the invention unless otherwise claimed. Any statement herein as to the nature or benefits of the invention or of the preferred embodiments is not intended to be limiting, and the appended claims should not be deemed to be limited by such statements. More generally, no language in the specification should be construed as indicating any non-claimed element as being essential to the practice of the invention. This invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contraindicated by context.

### LITERATURE CITED

1. Ahn J H, Choi Y S, Choi J H. Leptin promotes human endometriotic cell migration and invasion by up-regulating MMP-2 through the JAK2/STAT3 signaling pathway. Mol Hum Reprod 2015; 21:792-802.
2. Al-Hadiya B M. Niclosamide: comprehensive profile. Profiles Drug Subst Excip Relat Methodol 2005; 32:67-96.
3. Al Kadri H, Hassan S, Al-Fozan H M, Hajeer A. Hormone therapy for endometriosis and surgical menopause. Cochrane Database Syst Rev 2009:CD005997.
4. Andrews P, Thyssen J, Lorke D. The biology and toxicology of molluscicides, Bayluscide. Pharmacol Ther 1982; 19:245-295.
5. Attar E, Tokunaga H, Imir G, Yilmaz M B, Redwine D, Putman M, et al. Prostaglandin E2 via steroidogenic factor-1 coordinately regulates transcription of steroidogenic genes necessary for estrogen synthesis in endometriosis. J Clin Endocrinol Metab 2009; 94:623-631.
6. Berge, J. Pharmaceutical Salts. Pharm. Sci. 68(1):1-19 (1977).
7. Berkkanoglu M, Arici A. Immunology and endometriosis. Am J Reprod Immunol 2003; 50:48-59.
8. Bulun S E. Endometriosis. N Engl J Med 2009; 360:268-279.
9. Burney R O, Giudice L C. Pathogenesis and pathophysiology of endometriosis. Fertil Steril 2012; 98:511-519.
10. Capobianco A, Rovere-Querini P. Endometriosis, a disease of the macrophage. Front Immunol 2013; 4:9.
11. Chang Y, Yeh T, Lin K, Chen W, Yao H, Lan S, Wu Y, Hsieh H, Chen C, Chen C. Pharmacokinetics of Anti-SARS-CoV Agent Niclosamide and Its Analogs in Rats. J Food Drug Anal 2006; 14:329-333.
12. DeCherney A H. Endometriosis: recurrence and retreatment. Clin Ther 1992; 14:766-772; discussion 765.
13. Dennis G, Jr., Sherman B T, Hosack D A, Yang J, Gao W, Lane H C, et al. DAVID: Database for Annotation, Visualization, and Integrated Discovery. Genome Biol 2003; 4:P3.
14. Eskenazi B, Warner M L. Epidemiology of endometriosis. Obstet Gynecol Clin North Am 1997; 24:235-258.
15. Evers J L, Dunselman G A, Land J A, Bouckaert P X. Is there a solution for recurrent endometriosis? Br J Clin Pract Suppl 1991; 72:45-50; discussion 51-43.
16. Giudice L C, Kao L C. Endometriosis. Lancet 2004; 364:1789-1799.
17. Giudice L C. Clinical practice. Endometriosis. N Engl J Med 2010; 362:2389-2398.
18. Gonzalez-Ramos R, Defrere S, Devoto L. Nuclear factor-kappaB: a main regulator of inflammation and cell survival in endometriosis pathophysiology. Fertil Steril 2012a; 98:520-528.
19. Gonzalez-Ramos R, Rocco J, Rojas C, Sovino H, Poch A, Kohen P, et al. Physiologic activation of nuclear factor kappa-B in the endometrium during the menstrual cycle is altered in endometriosis patients. Fertil Steril 2012b; 97:645-651.
20. Guo S W. Recurrence of endometriosis and its control. Hum Reprod Update 2009; 15:441-461.
21. Han S J, Hawkins S M, Begum K, Jung S Y, Kovanci E, Qin J, et al. A new isoform of steroid receptor coactivator-1 is crucial for pathogenic progression of endometriosis. Nat Med 2012; 18:1102-1111.
22. Handbook of Pharmaceutical Excipients, Third Edition, American Pharmaceutical Association, USA and Pharmaceutical Press UK (2000), and Pharmaceutics—The Science of Dosage Form Design, Churchill Livingston (1988).
23. Hirata T, Osuga Y, Yoshino O, Hirota Y, Harada M, Takemura Y, et al. Development of an experimental model of endometriosis using mice that ubiquitously express green fluorescent protein. Hum Reprod 2005; 20:2092-2096.
24. Hoover, J. E., Remington's Pharmaceutical Sciences, Mack Publishing Co., Easton, Pa.; 1975.
25. Huang da W, Sherman B T, Lempicki R A. Systematic and integrative analysis of large gene lists using DAVID bioinformatics resources. Nat Protoc 2009; 4:44-57.
26. Itoh F, Komohara Y, Takaishi K, Honda R, Tashiro H, Kyo S, et al. Possible involvement of signal transducer and activator of transcription-3 in cell-cell interactions of peritoneal macrophages and endometrial stromal cells in human endometriosis. Fertil Steril 2013; 99:1705-1713.
27. Jin Y, Lu Z, Ding K, Li J, Du X, Chen C, et al. Antineoplastic mechanisms of niclosamide in acute myelogenous leukemia stem cells: inactivation of the NF-kappaB pathway and generation of reactive oxygen species. Cancer Res 2010; 70:2516-2527.
28. Kennedy S, Bergqvist A, Chapron C, D'Hooghe T, Dunselman G, Greb R, et al. Endometriosis ESIGf, Endometrium Guideline Development G. ESHRE guideline for the diagnosis and treatment of endometriosis. Hum Reprod 2005; 20:2698-2704.
29. Ketola K, Hilvo M, Hyotylainen T, Vuoristo A, Ruskeepaa A L, Oresic M, Kallioniemi O, Iljin K. Salinomycin inhibits prostate cancer growth and migration via induction of oxidative stress. Br J Cancer 2012; 106:99-106.
30. Khanim F L, Merrick B A, Giles H V, Jankute M, Jackson J B, Giles L J, et al. Redeployment-based drug screening identifies the anti-helminthic niclosamide as anti-myeloma therapy that also reduces free light chain production. Blood Cancer J 2011; 1:e39.
31. Kim B G, Yoo J Y, Kim T H, Shin J H, Langenheim J F, Ferguson S D, et al. Aberrant activation of signal transducer and activator of transcription-3 (STAT3) signaling in endometriosis. Hum Reprod 2015; 30:1069-1078.
32. Kim J J, Kurita T, Bulun S E. Progesterone action in endometrial cancer, endometriosis, uterine fibroids, and breast cancer. Endocr Rev 2013a; 34:130-162.
33. Kim S Y, Kang J W, Song X, Kim B K, Yoo Y D, Kwon Y T, et al. Role of the IL-6-JAK1-STAT3-Oct-4 pathway in the conversion of non-stem cancer cells into cancer stem-like cells. Cell Signal 2013b; 25:961-969.
34. King M L, Lindberg M E, Stodden G R, Okuda H, Ebers S D, Johnson A, et al. WNT7A/beta-catenin signaling induces FGF1 and influences sensitivity to niclosamide in ovarian cancer. Oncogene 2015; 34:3452-3462.
35. Kulak J, Jr., Fischer C, Komm B, Taylor H S. Treatment with bazedoxifene, a selective estrogen receptor modulator, causes regression of endometriosis in a mouse model. Endocrinology 2011; 152:3226-3232.
36. Li R, Hu Z, Sun S Y, Chen Z G, Owonikoko T K, Sica G L, Ramalingam S S, Curran W J, Khuri F R, Deng X. Niclosamide overcomes acquired resistance to erlotinib through suppression of STAT3 in non-small cell lung cancer. Mol Cancer Ther 2013a; 12:2200-2212.
37. Li R, You S, Hu Z, Chen Z G, Sica G L, Khuri F R, et al. Inhibition of STAT3 by niclosamide synergizes with erlotinib against head and neck cancer. PLoS One 2013b; 8:e74670.
38. Liberman, H. A. and Lachman, L., Eds., Pharmaceutical Dosage Forms, Marcel Decker, New York, N.Y., 1980.
39. Lima-Couy I, Cervero A, Bonilla-Musoles F, Pellicer A, Simon C. Endometrial leptin and leptin receptor expression in women with severe/moderate endometriosis. Mol Hum Reprod 2004; 10:777-782.
40. Matarese G, Alviggi C, Sanna V, Howard J K, Lord G M, Carravetta C, et al. Increased leptin levels in serum and peritoneal fluid of patients with pelvic endometriosis. J Clin Endocrinol Metab 2000; 85:2483-2487.
41. Meuleman C, Vandenabeele B, Fieuws S, Spiessens C, Timmerman D, D'Hooghe T. High prevalence of endometriosis in infertile women with normal ovulation and normospermic partners. Fertil Steril 2009; 92:68-74.
42. Molecular Cloning: A Laboratory Manual, 3rd ed., vol. 1-3, ed. Sambrook et al., Cold Spring Harbor Laboratory Press, Cold Spring Harbor, N.Y., 2001; and Current Protocols in Molecular Biology, ed. Ausubel et al., Greene Publishing and Wiley-Interscience, New York, 1992 (with periodic updates).
43. Oh H K, Choi Y S, Yang Y I, Kim J H, Leung P C, Choi J H. Leptin receptor is induced in endometriosis and leptin stimulates the growth of endometriotic epithelial cells through the JAK2/STAT3 and ERK pathways. Mol Hum Reprod 2013; 19:160-168.
44. Okamoto M, Nasu K, Abe W, Aoyagi Y, Kawano Y, Kai K, et al. Enhanced miR-210 expression promotes the pathogenesis of endometriosis through activation of signal transducer and activator of transcription 3. Hum Reprod 2015; 30:632-641.
45. ORGANIZATION W H, GENEVA. WHO SPECIFICATIONS AND EVALUATIONS FOR PUBLIC HEALTH PESTICIDES NICLOSAMIDE 2′,5-dichloro-4′-nitrosalicylanilide. In; 2002.
46. Osada T, Chen M, Yang X Y, Spasojevic I, Vandeusen J B, Hsu D, et al. Antihelminth compound niclosamide downregulates Wnt signaling and elicits antitumor responses in tumors with activating APC mutations. Cancer Res 2011; 71:4172-4182.
47. Pelch K E, Sharpe-Timms K L, Nagel S C. Mouse model of surgically-induced endometriosis by auto-transplantation of uterine tissue. J Vis Exp 2012:e3396.
48. Practice Committee of American Society for Reproductive M. Treatment of pelvic pain associated with endometriosis. Fertil Steril 2008; 90:S260-269.
49. Prather G R, MacLean J A, Shi M, Boadu D K, Paquet M, Hayashi K. Niclosamide As a Potential Nonsteroidal Therapy for Endometriosis That Preserves Reproductive Function in an Experimental Mouse Model. Biol Reprod 2016; 95:76-86.
50. Rana N, Braun D P, House R, Gebel H, Rotman C, Dmowski W P. Basal and stimulated secretion of cytokines by peritoneal macrophages in women with endometriosis. Fertil Steril 1996; 65:925-930.
51. Reardon S N, King M L, MacLean J A, 2nd, Mann J L, DeMayo F J, Lydon J P, et al. CDH1 is essential for endometrial differentiation, gland development, and adult function in the mouse uterus. Biol Reprod 2012; 86:141, 141-110.
52. Sack U, Walther W, Scudiero D, Selby M, Kobelt D, Lemm M, et al. Novel effect of antihelminthic Niclosamide on S100A4-mediated metastatic progression in colon cancer. J Natl Cancer Inst 2011; 103:1018-1036.
53. Satpathy M, Cao L, Pincheira R, Emerson R, Bigsby R, Nakshatri H, et al. Enhanced peritoneal ovarian tumor dissemination by tissue transglutaminase. Cancer Res 2007; 67:7194-7202.
54. Schaefer B C, Schaefer M L, Kappler J W, Marrack P, Kedl R M. Observation of antigen-dependent CD8+ T-cell/dendritic cell interactions in vivo. Cell Immunol 2001; 214:110-122.
55. Sheng Y H, Triyana S, Wang R, Das I, Gerloff K, Florin T H, et al. MUC1 and MUC13 differentially regulate epithelial inflammation in response to inflammatory and infectious stimuli. Mucosal Immunol 2013; 6:557-568.
56. Styer A K, Sullivan B T, Puder M, Arsenault D, Petrozza J C, Serikawa T, et al. Ablation of leptin signaling disrupts the establishment, development, and maintenance of endometriosis-like lesions in a murine model. Endocrinology 2008; 149:506-514.
57. Vigano P, Somigliana E, Matrone R, Dubini A, Barron C, Vignali M, et al. Serum leptin concentrations in endometriosis. J Clin Endocrinol Metab 2002; 87:1085-1087.
58. Waller K G, Shaw R W. Gonadotropin-releasing hormone analogues for the treatment of endometriosis: long-term follow-up. Fertil Steril 1993; 59:511-515.
59. Wieland A, Trageser D, Gogolok S, Reinartz R, Hofer H, Keller M, et al. Anticancer effects of niclosamide in human glioblastoma. Clin Cancer Res 2013; 19:4124-4136.
60. Weinbach E C, Garbus J. Mechanism of action of reagents that uncouple oxidative phosphorylation. Nature 1969; 221:1016-1018.
61. Wu M H, Chuang P C, Chen H M, Lin C C, Tsai S J. Increased leptin expression in endometriosis cells is associated with endometrial stromal cell proliferation and leptin gene up-regulation. Mol Hum Reprod 2002; 8:456-464.
62. Wu M H, Huang M F, Chang F M, Tsai S J. Leptin on peritoneal macrophages of patients with endometriosis. Am J Reprod Immunol 2010; 63:214-221.
63. Yoshioka S, King M L, Ran S, Okuda H, MacLean J A, 2nd, McAsey M E, et al. WNT7A regulates tumor growth and progression in ovarian cancer through the WNT/beta-catenin pathway. Mol Cancer Res 2012; 10:469-482.
64. Zhao Y, Li Q, Katzenellenbogen B S, Lau L F, Taylor R N, Bagchi I C, et al. Estrogen-induced CCN1 is critical for establishment of endometriosis-like lesions in mice. Mol Endocrinol 2014; 28:1934-1947.

1. A method for treating endometriosis comprising administering to a subject in need thereof a therapeutically effective amount of niclosamide or a pharmaceutically acceptable salt thereof.

2. The method of claim 1, wherein the subject is a mammal.

3. The method of claim 1, wherein the subject is pregnant or capable of becoming pregnant.

4. The method of claim 1, wherein the subject is an adolescent or adult human subject.

5. The method of claim 1, wherein the method is associated with reduction of endometrial lesions in the subject.

6. The method of claim 1, wherein the method is associated with reduction of endometrial pain in the subject.

7. The method according to claim 1 wherein a treatment of endometriosis is associated with reducing one or more symptoms associated with endometriosis.

8. The method of claim 1, wherein the niclosamide is administered orally.

9. The method of claim 8, which comprises orally administering the niclosamide in the form of a capsule, a sachet, a tablet, a syrup, an elixir, or a lozenge.

10. The method of claim 1, wherein the niclosamide is administered in a daily dosage of about 100-200 mg/kg of body weight.

11. The method of claim 1, wherein the niclosamide is administered in a daily dosage of about 200 mg/kg of body weight.

12. The method of claim 10, wherein the niclosamide is administered to the subject for at least three weeks.

13. A method for the treatment of endometriosis which comprises administering niclosamide or a pharmaceutically acceptable salt thereof to a subject in an amount sufficient to reduce or eliminate endometrial tissue outside the uterine cavity of the subject.

14. The method of treatment according to claim 13, wherein the treatment results in significant reduction in the expression of MKI67, but does not result in significant reduction of the expression of estrogen receptor or progesterone receptors, in endometrial lesions.

15. The method of treatment according to claim 13, wherein the treatment results in significant reduction in the expression of p-CHUK, p-STAT3, and NOS2, but does not result in significant reduction of the expression of COX2, in endometrial lesions.

16. The method of treatment according to claim 13, wherein the treatment inhibits the expression of p-CHUK, p-STAT3, and/or NFKB or STAT3 in endometrial lesions.

17. (canceled)

18. A compound of formula I: 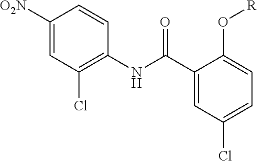
wherein R comprises at least one of CH₂)₂NH₃⁺M; (CH₂)₂N⁺(CH₃)₃M; (CH₂)₂SO₃⁻M; (CH₂)₂PO₃⁻M; CH₂CH₂(OCH₂CH₂)₃OH; or COCH₂(OCH₂CH₂)₃OH
and
wherein M comprises a pharmaceutically acceptable salt.

19. The compound of claim 18, wherein the compound comprises: 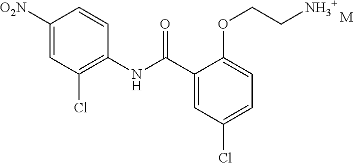
wherein M comprises a pharmaceutically acceptable salt.

20. The compound of claim 18, wherein M comprises Cl⁻.

21. The compound of claim 18, wherein R comprises at least one of: 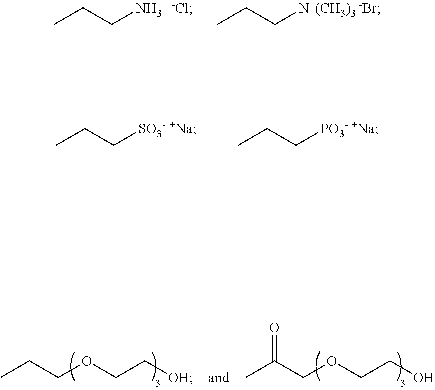
or mixtures thereof.

22. The compound of claim 18, wherein the compound has a water solubility greater than about 1 mM.

23. A medicament comprising the compound of claim 18.

24. The medicament of claim 23, wherein the compound is in a solid dosage unit suitable for oral administration.

25. (canceled)

26. (canceled)

27. (canceled)

28. A method for treating endometriosis comprising administering to a subject in need thereof a therapeutically effective amount of the compound of claim 18.

29. The method of claim 28, wherein the method is associated with reduction of endometrial lesions in the subject.

30. The method of claim 28, wherein the method is associated with reduction of endometrial pain in the subject.

31. A method for the treatment of endometriosis which comprises administering a compound of claim 18 to a subject in an amount sufficient to reduce or eliminate endometrial tissue outside the uterine cavity of the subject.

32. The method of claim 1, wherein the treatment does not disrupt reproductive function in the subject.

33. The method of claim 31, wherein the treatment does not disrupt reproductive function in the subject.
